(19) United States
(12) Patent Application Publication
(10) Pub. No.: US 2006/0167017 A1
(43) Pub. Date: Jul. 27, 2006

(54) Fungicidal triazolopyrimidines, method for the production thereof, use thereof for controlling harmful fungl, and agents containing said fungicadal triazolopyrimidines

(75) Inventors: Bernd Muller, Frankenthal (DE); Jordi Tormo I Blasco, Laudenbach (DE); Thomas Grote, Wachenheim (DE); Carsten Blettner, Mannheim (DE); Markus Gewehr, Kastellaun (DE); Wassilios Grammenos, Ludwigshafen (DE); Andreas Gypser, Mannheim (DE); Joachim Rheinheimer, Ludwigshafen (DE); Peter Schafer, Ottersheim (DE); Frank Schieweck, Hebheim (DE); Anja Schwogler, Mannheim (DE); Eberhard Ammermann, Heppenheim (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 10/539,039

(22) PCT Filed: Dec. 16, 2003
(86) PCT No.: PCT/EP03/14283

§ 371 Date: Jan. 3, 2006

(30) Foreign Application Priority Data
Dec. 17, 2002 (DE) 10259268.3

(51) Int. Cl. C07D 487/04; A01N 43/90

(52) U.S. Cl. 514/259.31; 544/256

(57) ABSTRACT

Triazolopyrimidines of the formula I 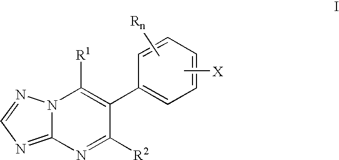 where the index and the substituents are as defined below:
R¹ is C₁-C₁₀-alkyl, C₂-C₁₀-alkenyl, C₂-C₁₀-alkynyl, C₃-C₁₀-cycloalkyl, C₃-C₁₀-cycloalkenyl, phenyl, naphthyl, or a five- to ten-membered saturated, partially unsaturated or aromatic heterocycle which is attached via carbon to the triazolopyrimidine and contains one to four heteroatoms from the group consisting of N, O and S;
R² is C₁-C₄-alkyl which may be substituted by halogen, cyano, nitro or C₁-C₂-alkoxy; n is 0 or an integer from 1 to 4; R is as defined in the description;
X is SOₘ—R^{x}, NR^{x}R^{y} or NR^{x}—(C═O)—R^{y}; m is 0 or an integer from 1 to 3; and processes for preparing these compounds, compositions comprising them and their use for controlling harmful fungi are described.

The present invention relates to triazolopyrimidines of the formula I 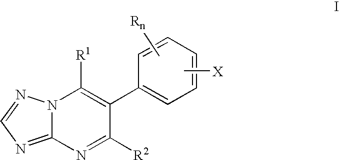
where the index and the substituents are as defined below:
R¹ is C₁-C₁₀-alkyl, C₂-C₁₀-alkenyl, C₂-C₁₀-alkynyl, C₃-C₁₀-cycloalkyl, C₃-C₁₀-cycloalkenyl, phenyl, naphthyl or a five- to ten-membered saturated, partially unsaturated or aromatic heterocycle which is attached via carbon to the triazolopyrimidine and contains one to four heteroatoms from the group consisting of O, N and S,
   where R¹ may be partially or fully halogenated or substituted by one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₃-C₆-cycloalkyl, phenyl, naphthyl, a five- to ten-membered saturated, partially unsaturated or aromatic heterocycle which contains one to four heteroatoms from the group consisting of O, N and S, where these aliphatic, alicyclic or aromatic groups for their part may be partially or fully halogenated or carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkynyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the abovementioned alkenyl or alkynyl groups in these radicals contain 2 to 8 carbon atoms and the abovementioned groups may be partially or fully halogenated;
   and/or one to three of the following radicals:
   cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups;
R² is C₁-C₄-alkyl which may be substituted by halogen, cyano, nitro or C₁-C₂-alkoxy;
n is 0 or an integer from 1 to 4;
R is halogen, cyano, C₁-C₆-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₁-C₈-alkoxycarbonyl, C₂-C₁₀-alkenyloxycarbonyl, C₂-C₁₀-alkynyloxycarbonyl, aminocarbonyl, C₁-C₈-alkylaminocarbonyl, di-(C₁-C₈-)alkylaminocarbonyl, C₁-C₈-alkoximinoalkyl, C₂-C₁₀-alkenyloximinoalkyl, C₂-C₁₀-alkynyloximinoalkyl, C₁-C₈-alkylcarbonyl, C₂-C₁₀-alkenylcarbonyl, C₂-C₁₀-alkynylcarbonyl, C₃-C₆-cycloalkylcarbonyl, or a five- to ten-membered saturated, partially unsaturated or aromatic heterocycle which contains one to four heteroatoms from the group consisting of O, N and S;
X is SOₘ—R^{x}, NR^{x}R^{y} or NR^{x}—(C═O)—R^{y};
   R^{X}, R^{Y} are: hydrogen, C₁-C₆-alkyl, C₂-C₁₀-alkenyl, C₂-C₁₀-alkynyl, C₃-C₆-cycloalkyl, C₃-C₆-cycloalkenyl, where the above radicals may be partially or fully halogenated or substituted by cyano, C₁-C₄-alkoximino, C₂-C₄-alkenyloximino, C₂-C₄-alkynyloximino or C₁-C₄-alkoxy;
m is 0 or an integer 1 to 3.

Moreover, the invention relates to a process for preparing these compounds, to compositions comprising them and to their use for controlling harmful fungi.

5-chlorotriazolopyrimidines for controlling harmful fungi are disclosed in EP-A 71 792, EP-A 550 113, WO-A 94/20501, EP-A 834 513, WO-A 98/46608 and WO-A 99/41255.

However, in many cases, their activity is unsatisfactory. It is an object of the invention to provide compounds having improved activity.

We have found that this object is achieved by the compounds defined at the outset. Furthermore, we have found processes for their preparation, compositions comprising them and methods for controlling harmful fungi using the compounds I.

The compounds of the formula I differ from the compounds in the abovementioned publications in that the 5-alkyl radical is combined with groups in position 7 which are attached via carbon.

Compared to the known compounds, the compounds of the formula I have increased activity against harmful fungi.

The compounds I can be obtained by different routes; advantageously, 5-aminotriazole of the formula II is used as starting material and condensed with dicarbonyl compounds of the formula III.

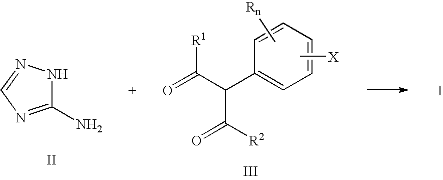

This reaction usually takes place at temperatures of from 80° C. to 250° C., preferably from 120° C. to 180° C., in the absence of a solvent or in an inert organic solvent in the presence of a base [cf. EP-A 770 615] or in the presence of acetic acid under the conditions known from Adv. Het. Chem. 57 (1993), 81 ff.

Suitable solvents are aliphatic hydrocarbons, aromatic hydrocarbons such as toluene, o-, m- and p-xylene, halogenated hydrocarbons, ethers, nitriles, ketones, alcohols, and also N-methylpyrrolidone, dimethyl sulfoxide, dimethylformamide and dimethylacetamide. The reaction is particularly preferably carried out in the absence of a solvent or in ethylene glycol dimethyl ether, chlorobenzene, xylene, dimethyl sulfoxide or N-methylpyrrolidone. It is also possible to use mixtures of the solvents mentioned.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, diisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to tertiary amines such as triisopropylamine, tributylamine, N-methylmorpholine or N-methylpiperidine.

The bases are generally employed in catalytic amounts; however, they can also be employed in equimolar amounts, in excess or, if appropriate, as solvent.

The starting materials are generally reacted with one another in equimolar amounts. In terms of yield, it may be advantageous to employ an excess of base and diketone III, based on II.

The diketones III can be prepared analogously to processes known from the literature, for example as described in the publications mentioned above. Diketones having an acylamino substituent can be obtained, for example, by acylation from the corresponding amino group compound. In general, the amino grouping can be introduced into the phenyl ring by reducing a suitable nitro precursor. The sulfonic acid grouping can be introduced into the phenyl ring by direct sulfonylation of a suitable precursor using sulfuric acid or oleum. However, the sulfonic acid grouping can also be synthesized from a suitable diazonium salt by Sandmeyer reaction with sulfur trioxide. The diazonium salt can be obtained from the abovementioned amino compound. The sulfoxides and sulfones can be prepared by oxidizing the corresponding alkyl aryl sulfides by processes known from the literature using, for example, hydrogen peroxide, peracids or selenium dioxide.

Compounds of the formula I can also be obtained by coupling 5-halotriazolopyrimidines of the formula IV (Y is halogen, in particular chlorine or bromine) with organometallic reagents of the formula VII.

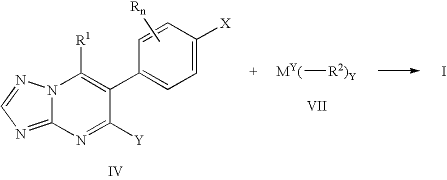

In the formula VII, M is a metal ion having the valency Y, such as, for example, B, Zn, Mg or Sn. In one embodiment of this process, the reaction is carried out with transition metal catalysis, such as Ni or Pd catalysis. This reaction can be carried out, for example, analogously to the following methods: J. Chem. Soc. Perkin Trans. 1 (1994), 1187, ibid (1996), 2345; WO-A 99/41255; Aust. J. Chem. 43 (1990), 733; J. Org. Chem. 43 (1978), 358; J. Chem. Soc. Chem. Commun. (1979), 866; Tetrahedron Lett. 34 (1993), 8267; ibid 33 (1992), 413. In particular in cases where M is Zn or Mg, the reaction can also be carried out in the absence of a catalyst. The compounds IV are known from the publications cited at the outset. They are obtained, in particular, from 5,7-dichlorotriazolopyrimidines by introducing the radical R¹ using organometallic processes similar to those described above.

The compounds of the formula I′ according to the invention can also be obtained by reacting 5-halotriazolopyrimidines of the formula IV with substituted malonic acid esters of the formula V, where Rx is C₁-C₄-alkyl, allyl, phenyl or benzyl, followed by hydrolysis of the resulting ester VI and decarboxylation of the carboxylic acid VIa.

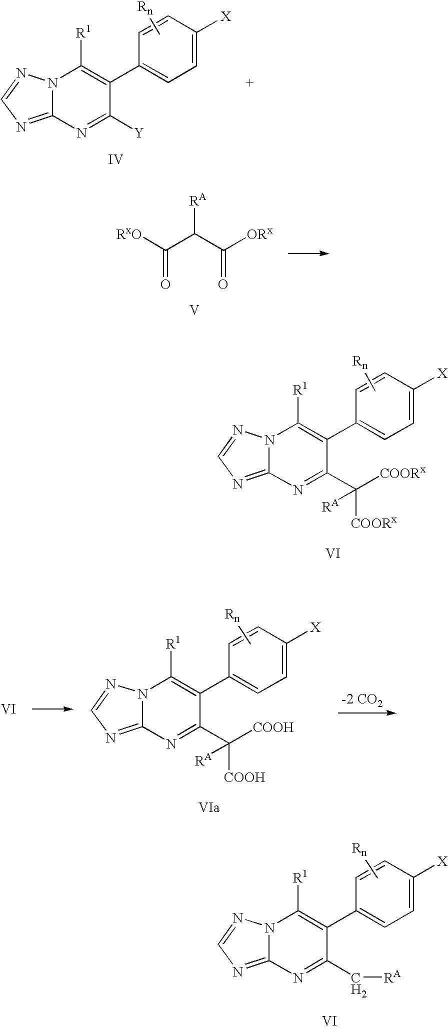

In the formula IV, Y is halogen, in particular chlorine or bromine. The compounds IV are known from the publications cited at the outset. In the formula I′, n, R and R¹ have the definitions given for the formula I and R^{A} is hydrogen or C₁-C₃-alkyl which may be substituted by halogen, cyano, nitro or C₁-C₂-alkoxy.

In a preferred embodiment of the process according to the invention, R^{A} is hydrogen or methyl, in particular hydrogen.

The starting materials V are known from the literature [J. Am. Chem. Soc. 64 (1942), 2714; J. Org. Chem. 39 (1974), 2172; Helv. Chim. Acta 61 (1978), 1565], or they can be prepared according to the literature cited.

The subsequent hydrolysis of the ester is carried out under generally known conditions [cf.: Greene & Wuts, Protective Groups in Organic Synthesis, Wiley (1991), p. 224 ff.: Cleavage of alkyl esters under Pd catalysis (p. 248); hydrolysis of benzyl esters (p. 251); Cleavage of methyl or ethyl esters in the presence of lithium salts such as LiI (p. 232), LiBr or LiCl; or under acidic or alkaline conditions]. Depending on the structural elements R^{A}, Rₙ and R¹, alkaline or acidic hydrolysis of the compounds VI may be advantageous. It is possible that full or partial decarboxylation to I′ takes place even under the conditions of ester hydrolysis.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; the reaction is particularly preferably carried out in hydrochloric acid or acetic acid. It is also possible to use mixtures of the solvents mentioned.

The reaction mixtures are worked up in a customary manner, for example by mixing with water, phase separation and, if required, chromatographic purification of the crude products. Some of the intermediates and end products are obtained in the form of colorless or slightly brownish, viscous oils, which are purified or freed from volatile components under reduced pressure and at moderately elevated temperatures. If the intermediates and end products are obtained as solids, purification can also be carried out by recrystallization or digestion.

If individual compounds I cannot be obtained by the routes described above, they can be prepared by derivatization of other compounds I.

If the synthesis yields isomer mixtures, a separation is generally not necessarily required since in some cases the individual isomers can be converted into one another during the preparation for use or upon use (for example under the action of light, acids or bases). Similar conversions may also occur after use, for example in the treatment of plants in the treated plant or in the harmful fungus or animal pest to be controlled.

In the definitions of the symbols given in the above formulae, collective terms were used which generally represent the following substituents:

halogen: fluorine, chlorine, bromine and iodine;

alkyl: saturated, straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 10 carbon atoms (as mentioned above), where all or some of the hydrogen atoms in these groups may be replaced by halogen atoms as mentioned above, for example C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

alkenyl: unsaturated, straight-chain or branched hydrocarbon radicals having 2 to 4, 6, 8 or 10 carbon atoms and a double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: unsaturated, straight-chain or branched hydrocarbon radicals having 2 to 10 carbon atoms and a double bond in any position (as mentioned above), where all or some of the hydrogen atoms in these groups may be replaced by halogen atoms as entioned above, in particular by fluorine, chlorine and bromine;

alkynyl: straight-chain or branched hydrocarbon groups having 2 to 4, 6, 8 or 10 carbon atoms and a triple bond in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic, saturated hydrocarbon groups having 3 to 6, 8, 10 or 12 carbon ring members, for example C₃-C₈-cycloalkyl such as cyclopropyl, cyclobutyl, cyclopentyl, cyclohexyl, cycloheptyl and cyclooctyl, or C₇-C₁₂-bicycloalkyl;

aryl: a mono- to trinuclear aromatic ring system comprising 6 to 14 carbon ring members, for example phenyl, naphthyl and anthracenyl;

five- to ten-membered saturated, partially unsaturated or aromatic heterocycle which is bonded to triazolopyrimidine via carbon and contains one to four heteroatoms from the group consisting of O, N and S:

5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydro-triazin-2-yl and 1,2,4-hexahydrotriazin-3-yl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
benzo-fused 5-membered heteroaryl which contains one to three nitrogen atoms or one nitrogen atom and one oxygen or sulfur atom: 5-membered heteroaryl groups which, in addition to carbon atoms, may contain one to four nitrogen atoms or one to three nitrogen atoms and one sulfur or oxygen atom as ring members and in which two adjacent carbon ring members or a nitrogen and an adjacent carbon ring member may be bridged by a buta-1,3-diene-1,4-diyl group;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The scope of the present invention includes the (R) and (S) isomers and the racemates of compounds of the formula I having chiral centers.

With a view to the intended use of the triazolopyrimidines of the formula I, the following meanings of the substituents are particularly preferred, in each case on their own or in combination:

Preference is given to compounds I in which R¹ is C₃-C₈-alkyl, C₃-C₈-alkenyl, C₃-C₈-alkynyl, C₃-C₆-cycloalkyl or C₅-C₆-cycloalkenyl.

Particular preference is given to compounds I in which R¹ is C₁-C₈-alkyl or C₁-C₆-haloalkyl.

Additionally, preference is given to compounds I in which R¹ is C₂-C₁₀-alkynyl and in particular C₂-C₁₀-alkenyl. Particular preference is given to branched C₂-C₁₀-alkenyl.

Preference is likewise given to compounds I in which R¹ is a 5- or 6-membered saturated or aromatic heterocycle which is attached via carbon.

Moreover, particular preference is given to compounds I in which R¹ is C₃-C₆-cycloalkyl or C₅-C₆-cycloalkyl which may be substituted by C₁-C₄-alkyl.

Particular preference is given to compounds I in which R^{a} is halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino, C₂-C₆-alkynyloximino, C₃-C₆-cycloalkyl or C₅-C₆-cycloalkenyl, where the aliphatic or alicyclic groups for their part may be partially or fully halogenated or may carry one to three groups R^{b}.

Particular preference is given to compounds I in which R^{b} is halogen, cyano, C₁-C₆-alkyl, C₂-C₆-alkenyl, C₂-C₆-alkynyl, C₁-C₆-alkylcarbonyl, C₁-C₆-haloalkylcarbonyl or C₁-C₆-alkoxy.

Particular preference is also given to compounds I in which R² is C₁-C₄-alkyl which may be substituted by halogen.

Particular preference is likewise given to compounds I in which R² is methyl.

In addition, preference is given to compounds I in which R² is halomethyl.

Particular preference is also given to compounds I in which a substituent R is located in the 2-position and n is an integer from 1 to 3, in particular 1 or 2.

Moreover, particular preference is given to compounds I in which n is 2 or 3 and a substituent R is located in the 2-position.

Preference is furthermore given to compounds I in which R is fluorine, chlorine, bromine, cyano, C₁-C₆-alkyl or C₁-C₆-alkoxy.

Particular preference is likewise given to compounds I in which R is fluorine, chlorine, methyl, trifluoromethyl or methoxy.

In addition, particular preference is given to compounds I in which Rₙ is 2-chloro, 2-fluoro, 2-methyl, 2-methoxy, 2-trifluoromethyl, 2-trifluoromethyl-6-chloro, 2-chloro-6-fluoro, 2,6-difluoro, 2-fluoro-6-methyl, 2,4-difluoro, 2-fluoro-4-chloro, 2-fluoro-3-methyl, 2-fluoro-4-methyl, 2-chloro-4-fluoro, 2,4-dichloro, 2-chloro-4-methyl, 2-chloro-3-methyl, 2,6-dichloro, 2-chloro-6-methyl, 2-methyl-4-fluoro, 2-methyl-4-chloro, 2,4-dimethyl, 2,3-dimethyl, 2-methyl-6-fluoro, 2-methyl-6-chloro or 2,6-dimethyl.

Moreover, particular preference is given to compounds I in which X is C₁-C₆-alkylsulfonyl, C₁-C₆-alkylsulfenyl, C₁-C₆-alkylsulfoxyl, C₁-C₆-alkylmercapto, amino, C₁-C₆-alkylamino, di-(C₁-C₆-alkyl)amino, C₁-C₆-alkylcarbonylamino, C₁-C₆-alkylcarbonyl (C₁-C₆-alkyl) amino.

Preference is given to compounds I in which the substituent X is located in the 3- or 5-position on the phenyl ring.

Especially preferred are compounds I in which the substituent X is located in the 4-position on the phenyl ring.

Particular preference is likewise given to compounds I in which m is 1 or 2. The sulfur atom is preferably attached directly to the phenyl ring. If m is 2 or 3, the sulfur may also be attached to the phenyl ring via oxygen.

Particular preference is given to triazolopyrimidines of the formula I′ 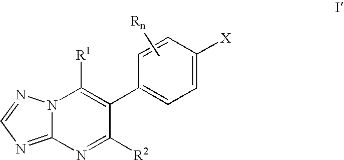
where the index and the substituents are as defined below:
R¹ is C₃-C₈-alkyl, C₃-C₈-alkenyl, C₃-C₈-alkynyl, C₃-C₆-cycloalkyl, C₅-C₆-cycloalkenyl; where R¹ may be partially or fully halogenated or substituted by one to four identical or different groups R^{a}:
   R^{a} is halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino, C₂-C₆-alkynyloximino;
R² is C₁-C₄-alkyl which may be substituted by halogen;
n is an integer from 0 to 2;
R is fluorine, chlorine, bromine, cyano, C₁-C₆-alkyl, C₁-C₆-alkoxy;
X is SO—R^{x}, SO₂—R^{x} or NR^{x}—(C═O)—R^{y};
   R^{x}, R^{y} are: hydrogen, C₁-C₆-alkyl, C₂-C₆-alkenyl or C₃-C₆-cycloalkyl, where the above radicals may be partially or fully halogenated.

With a view to their use, most particular preference is given to the compounds IA compiled in the tables below. Moroever, the groups mentioned in the tables for a substituent are themselves, independently of the combination in which they are mentioned, a particularly preferred embodiment of the substituent in question. 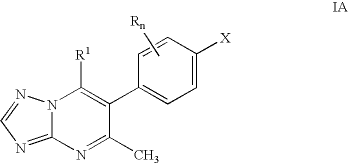
Table 1

Compounds of the formula IA in which Rₙ is 2-chloro, X is acetylamino and R¹ for each compound corresponds to one row of table A

Table 2

Compounds of the formula IA in which Rₙ is 2-fluoro, X is acetylamino and R¹ for each compound corresponds to one row of table A

Table 3

Compounds of the formula IA in which Rₙ is 2-methyl, X is acetylamino and R¹ for each compound corresponds to one row of table A

Table 4

Compounds of the formula IA, in which Rₙ is 2,6-dichloro, X is acetylamino and R¹ for each compound corresponds to one row of table A

Table 5

Compounds of the formula IA, in which Rₙ is 2,6-difluoro, X is acetylamino and R¹ for each compound corresponds to one row of table A

Table 6

Compounds of the formula IA, in which Rₙ is 2,6-dimethyl, X is acetylamino and R¹ for each compound corresponds to one row of table A

Table 7

Compounds of the formula IA, in which Rₙ is 2-chloro-3-methyl, X is acetylamino and R¹ for each compound corresponds to one row of table A

Table 8

Compounds of the formula IA, in which Rₙ is 2-fluoro-3-methyl, X is acetylamino and R¹ for each compound corresponds to one row of table A

Table 9

Compounds of the formula IA, in which Rₙ is 2,3-dimethyl, X is acetylamino and R¹ for each compound corresponds to one row of table A

Table 10

Compounds of the formula IA, in which Rₙ is 2-chloro-6-fluoro, X is acetylamino and R¹ for each compound corresponds to one row of table A

Table 11

Compounds of the formula IA, in which Rₙ is 2-chloro-6-methyl, x is acetylamino and R¹ for each compound corresponds to one row of table A

Table 12

Compounds of the formula IA, in which Rₙ is 2-fluoro-6-methyl, X is acetylamino and R¹ for each compound corresponds to one row of table A

Table 13

Compounds of the formula IA, in which Rₙ is 2-chloro, X is N-acetyl-N-methylamino and R¹ for each compound corresponds to one row of table A

Table 14

Compounds of the formula IA, in which Rₙ is 2-fluoro, X is N-acetyl-N-methylamino and R¹ for each compound corresponds to one row of table A

Table 15

Compounds of the formula IA, in which Rₙ is 2-methyl, X is N-acetyl-N-methylamino and R¹ for each compound corresponds to one row of table A

Table 16

Compounds of the formula IA, in which Rₙ is 2,6-dichloro, X is N-acetyl-N-methylamino and R¹ for each compound corresponds to one row of table A

Table 17

Compounds of the formula IA, in which Rₙ is 2,6-difluoro, X is N-acetyl-N-methylamino and R¹ for each compound corresponds to one row of table A

Table 18

Compounds of the formula IA, in which Rₙ is 2,6-dimethyl, X is N-acetyl-N-methylamino and R¹ for each compound corresponds to one row of table A

Table 19

Compounds of the formula IA, in which Rₙ is 2-chloro-3-methyl, X is N-acetyl-N-methylamino and R¹ for each compound corresponds to one row of table A

Table 20

Compounds of the formula IA, in which Rₙ is 2-fluoro-3-methyl, X is N-acetyl-N-methylamino and R¹ for each compound corresponds to one row of table A

Table 21

Compounds of the formula IA, in which Rₙ is 2,3-dimethyl, X is N-acetyl-N-methylamino and R¹ for each compound corresponds to one row of table A

Table 22

Compounds of the formula IA, in which Rₙ is 2-chloro-6-fluoro, X is N-acetyl-N-methylamino and R¹ for each compound corresponds to one row of table A

Table 23

Compounds of the formula IA, in which Rₙ is 2-chloro-6-methyl, X is N-acetyl-N-methylamino and R¹ for each compound corresponds to one row of table A

Table 24

Compounds of the formula IA, in which Rₙ is 2-fluoro-6-methyl, X is N-acetyl-N-methylamino and R¹ for each compound corresponds to one row of table A

Table 25

Compounds of the formula IA, in which Rₙ is 2-chloro, X is N-acetyl-N-ethylamino and R¹ for each compound corresponds to one row of table A

Table 26

Compounds of the formula IA, in which Rₙ is 2-fluoro, X is N-acetyl-N-ethylamino and R¹ for each compound corresponds to one row of table A

Table 27

Compounds of the formula IA, in which Rₙ is 2-methyl, X is N-acetyl-N-ethylamino and R¹ for each compound corresponds to one row of table A

Table 28

Compounds of the formula IA, in which Rₙ is 2,6-dichloro, X is N-acetyl-N-ethylamino and R¹ for each compound corresponds to one row of table A

Table 29

Compounds of the formula IA, in which Rₙ is 2,6-difluoro, X is N-acetyl-N-ethylamino and R¹ for each compound corresponds to one row of table A

Table 30

Compounds of the formula IA, in which Rₙ is 2,6-dimethyl, X is N-acetyl-N-ethylamino and R¹ for each compound corresponds to one row of table A

Table 31

Compounds of the formula IA, in which Rₙ is 2-chloro-3-methyl, X is N-acetyl-N-ethylamino and R¹ for each compound corresponds to one row of table A

Table 32

Compounds of the formula IA, in which Rₙ is 2-fluoro-3-methyl, X is N-acetyl-N-ethylamino and R¹ for each compound corresponds to one row of table A

Table 33

Compounds of the formula IA, in which Rₙ is 2,3-dimethyl, X is N-acetyl-N-ethylamino and R¹ for each compound corresponds to one row of table A

Table 34

Compounds of the formula IA, in which Rₙ is 2-chloro-6-fluoro, X is N-acetyl-N-ethylamino and R¹ for each compound corresponds to one row of table A

Table 35

Compounds of the formula IA, in which Rₙ is 2-chloro-6-methyl, X is N-acetyl-N-ethylamino and R¹ for each compound corresponds to one row of table A

Table 36

Compounds of the formula IA, in which Rₙ is 2-fluoro-6-methyl, X is N-acetyl-N-ethylamino and R¹ for each compound corresponds to one row of table A

Table 37

Compounds of the formula IA, in which Rₙ is 2-chloro, X is propionylamino and R¹ for each compound corresponds to one row of table A

Table 38

Compounds of the formula IA, in which Rₙ is 2-fluoro, X is propionylamino and R¹ for each compound corresponds to one row of table A

Table 39

Compounds of the formula IA, in which Rₙ is 2-methyl, X is propionylamino and R¹ for each compound corresponds to one row of table A

Table 40

Compounds of the formula IA, in which Rₙ is 2,6-dichloro, X is propionylamino and R¹ for each compound corresponds to one row of table A

Table 41

Compounds of the formula IA, in which Rₙ is 2,6-difluoro, X is propionylamino and R¹ for each compound corresponds to one row of table A

Table 42

Compounds of the formula IA, in which Rₙ is 2,6-dimethyl, X is propionylamino and R¹ for each compound corresponds to one row of table A

Table 43

Compounds of the formula IA, in which Rₙ is 2-chloro-3-methyl, X is propionylamino and R¹ for each compound corresponds to one row of table A

Table 44

Compounds of the formula IA, in which Rₙ is 2-fluoro-3-methyl, X is propionylamino and R¹ for each compound corresponds to one row of table A

Table 45

Compounds of the formula IA, in which Rₙ is 2,3-dimethyl, X is propionylamino and R¹ for each compound corresponds to one row of table A

Table 46

Compounds of the formula IA, in which Rₙ is 2-chloro-6-fluoro, X is propionylamino and R¹ for each compound corresponds to one row of table A

Table 47

Compounds of the formula IA, in which Rₙ is 2-chloro-6-methyl, X is propionylamino and R¹ for each compound corresponds to one row of table A

Table 48

Compounds of the formula IA, in which Rₙ is 2-fluoro-6-methyl, X is propionylamino and R¹ for each compound corresponds to one row of table A

Table 49

Compounds of the formula IA, in which Rₙ is 2-chloro, X is N-propionyl-N-methylamino and R¹ for each compound corresponds to one row of table A

Table 50

Compounds of the formula IA, in which Rₙ is 2-fluoro, X is N-propionyl-N-methylamino and R¹ for each compound corresponds to one row of table A

Table 51

Compounds of the formula IA, in which Rₙ is 2-methyl, X is N-propionyl-N-methylamino and R¹ for each compound corresponds to one row of table A

Table 52

Compounds of the formula IA, in which Rₙ is 2,6-dichloro, X is N-propionyl-N-methylamino and R¹ for each compound corresponds to one row of table A

Table 53

Compounds of the formula IA, in which Rₙ is 2,6-difluoro, X is N-propionyl-N-methylamino and R¹ for each compound corresponds to one row of table A

Table 54

Compounds of the formula IA, in which Rₙ is 2,6-dimethyl, X is N-propionyl-N-methylamino and R¹ for each compound corresponds to one row of table A Table 55

Compounds of the formula IA, in which Rₙ is 2-chloro-3-methyl, X is N-propionyl-N-methylamino and R¹ for each compound corresponds to one row of table A

Table 56

Compounds of the formula IA, in which Rₙ is 2-fluoro-3-methyl, X is N-propionyl-N-methylamino and R¹ for each compound corresponds to one row of table A

Table 57

Compounds of the formula IA, in which Rₙ is 2,3-dimethyl, X is N-propionyl-N-methylamino and R¹ for each compound corresponds to one row of table A

Table 58

Compounds of the formula IA, in which Rₙ is 2-chloro-6-fluoro, X is N-propionyl-N-methylamino and R¹ for each compound corresponds to one row of table A

Table 59

Compounds of the formula IA, in which Rₙ is 2-chloro-6-methyl, X is N-propionyl-N-methylamino and R¹ for each compound corresponds to one row of table A

Table 60

Compounds of the formula IA, in which Rₙ is 2-fluoro-6-methyl, X is N-propionyl-N-methylamino and R¹ for each compound corresponds to one row of table A

Table 61

Compounds of the formula IA, in which Rₙ is 2-chloro, X is N-propionyl-N-ethylamino and R¹ for each compound corresponds to one row of table A

Table 62

Compounds of the formula IA, in which Rₙ is 2-fluoro, X is N-propionyl-N-ethylamino and R¹ for each compound corresponds to one row of table A

Table 63

Compounds of the formula IA, in which Rₙ is 2-methyl, X is N-propionyl-N-ethylamino and R¹ for each compound corresponds to one row of table A

Table 64

Compounds of the formula IA, in which Rₙ is 2,6-dichloro, X is N-propionyl-N-ethylamino and R¹ for each compound corresponds to one row of table A

Table 65

Compounds of the formula IA, in which Rₙ is 2,6-difluoro, X is N-propionyl-N-ethylamino and R¹ for each compound corresponds to one row of table A

Table 66

Compounds of the formula IA, in which Rₙ is 2,6-dimethyl, X is N-propionyl-N-ethylamino and R¹ for each compound corresponds to one row of table A

Table 67

Compounds of the formula IA, in which Rₙ is 2-chloro-3-methyl, X is N-propionyl-N-ethylamino and R¹ for each compound corresponds to one row of table A

Table 68

Compounds of the formula IA, in which Rₙ is 2-fluoro-3-methyl, X is N-propionyl-N-ethylamino and R¹ for each compound corresponds to one row of table A

Table 69

Compounds of the formula IA, in which Rₙ is 2,3-dimethyl, X is N-propionyl-N-ethylamino and R¹ for each compound corresponds to one row of table A

Table 70

Compounds of the formula IA, in which Rₙ is 2-chloro-6-fluoro, X is N-propionyl-N-ethylamino and R¹ for each compound corresponds to one row of table A

Table 71

Compounds of the formula IA, in which Rₙ is 2-chloro-6-methyl, X is N-propionyl-N-ethylamino and R¹ for each compound corresponds to one row of table A

Table 72

Compounds of the formula IA, in which Rₙ is 2-fluoro-6-methyl, X is N-propionyl-N-ethylamino and R¹ for each compound corresponds to one row of table A

Table 73

Compounds of the formula IA, in which Rₙ is 2-chloro, X is 2-methylpropionylamino and R¹ for each compound corresponds to one row of table A

Table 74

Compounds of the formula IA, in which Rₙ is 2-fluoro, X is 2-methylpropionylamino and R¹ for each compound corresponds to one row of table A

Table 75

Compounds of the formula IA, in which Rₙ is 2-methyl, X is 2-methylpropionylamino and R¹ for each compound corresponds to one row of table A

Table 76

Compounds of the formula IA, in which Rₙ is 2,6-dichloro, X is 2-methylpropionylamino and R¹ for each compound corresponds to one row of table A

Table 77

Compounds of the formula IA, in which Rₙ is 2,6-difluoro, X is 2-methylpropionylamino and R¹ for each compound corresponds to one row of table A

Table 78

Compounds of the formula IA, in which Rₙ is 2,6-dimethyl, X is 2-methylpropionylamino and R¹ for each compound corresponds to one row of table A

Table 79

Compounds of the formula IA, in which Rₙ is 2-chloro-3-methyl, X is 2-methylpropionylamino and R¹ for each compound corresponds to one row of table A

Table 80

Compounds of the formula IA, in which Rₙ is 2-fluoro-3-methyl, X is 2-methylpropionylamino and R¹ for each compound corresponds to one row of table A

Table 81

Compounds of the formula IA, in which Rₙ is 2,3-dimethyl, X is 2-methylpropionylamino and R¹ for each compound corresponds to one row of table A

Table 82

Compounds of the formula IA, in which Rₙ is 2-chloro-6-fluoro, X is 2-methylpropionylamino and R¹ for each compound corresponds to one row of table A

Table 83

Compounds of the formula IA, in which Rₙ is 2-chloro-6-methyl, x is 2-methylpropionylamino and R¹ for each compound corresponds to one row of table A

Table 84

Compounds of the formula IA, in which Rₙ is 2-fluoro-6-methyl, X is 2-methylpropionylamino and R¹ for each compound corresponds to one row of table A

Table 85

Compounds of the formula IA, in which Rₙ is 2-chloro, X is N-2-methylpropionyl-N-methylamino and R¹ for each compound corresponds to one row of table A

Table 86

Compounds of the formula IA, in which Rₙ is 2-fluoro, X is N-2-methylpropionyl-N-methylamino and R¹ for each compound corresponds to one row of table A

Table 87

Compounds of the formula IA, in which Rₙ is 2-methyl, X is N-2-methylpropionyl-N-methylamino and R¹ for each compound corresponds to one row of table A

Table 88

Compounds of the formula IA, in which Rₙ is 2,6-dichloro, X is N-2-methylpropionyl-N-methylamino and R¹ for each compound corresponds to one row of table A

Table 89

Compounds of the formula IA, in which Rₙ is 2,6-difluoro, X is N-2-methylpropionyl-N-methylamino and R¹ for each compound corresponds to one row of table A

Table 90

Compounds of the formula IA, in which Rₙ is 2,6-dimethyl, X is N-2-methylpropionyl-N-methylamino and R¹ for each compound corresponds to one row of table A

Table 91

Compounds of the formula IA, in which Rₙ is 2-chloro-3-methyl, X is N-2-methylpropionyl-N-methylamino and R¹ for each compound corresponds to one row of table A

Table 92

Compounds of the formula IA, in which Rₙ is 2-fluoro-3-methyl, X is N-2-methylpropionyl-N-methylamino and R¹ for each compound corresponds to one row of table A

Table 93

Compounds of the formula IA, in which Rₙ is 2,3-dimethyl, X is N-2-methylpropionyl-N-methylamino and R¹ for each compound corresponds to one row of table A

Table 94

Compounds of the formula IA, in which Rₙ is 2-chloro-6-fluoro, X is N-2-methylpropionyl-N-methylamino and R¹ for each compound corresponds to one row of table A

Table 95

Compounds of the formula IA, in which Rₙ is 2-chloro-6-methyl, X is N-2-methylpropionyl-N-methylamino and R¹ for each compound corresponds to one row of table A

Table 96

Compounds of the formula IA, in which Rₙ is 2-fluoro-6-methyl, X is N-2-methylpropionyl-N-methylamino and R¹ for each compound corresponds to one row of table A

Table 97

Compounds of the formula IA, in which Rₙ is 2-chloro, X is N-2-methylpropionyl-N-ethylamino and R¹ for each compound corresponds to one row of table A

Table 98

Compounds of the formula IA, in which Rₙ is 2-fluoro, X is N-2-methylpropionyl-N-ethylamino and R¹ for each compound corresponds to one row of table A

Table 99

Compounds of the formula IA, in which Rₙ is 2-methyl, X is N-2-methylpropionyl-N-ethylamino and R¹ for each compound corresponds to one row of table A

Table 100

Compounds of the formula IA, in which Rₙ is 2,6-dichloro, X is N-2-methylpropionyl-N-ethylamino and R¹ for each compound corresponds to one row of table A

Table 101

Compounds of the formula IA, in which Rₙ is 2,6-difluoro, X is N-2-methylpropionyl-N-ethylamino and R¹ for each compound corresponds to one row of table A

Table 102

Compounds of the formula IA, in which Rₙ is 2,6-dimethyl, X is N-2-methylpropionyl-N-ethylamino and R¹ for each compound corresponds to one row of table A

Table 103

Compounds of the formula IA, in which Rₙ is 2-chloro-3-methyl, X is N-2-methylpropionyl-N-ethylamino and R¹ for each compound corresponds to one row of table A

Table 104

Compounds of the formula IA, in which Rₙ is 2-fluoro-3-methyl, X is N-2-methylpropionyl-N-ethylamino and R¹ for each compound corresponds to one row of table A

Table 105

Compounds of the formula IA, in which Rₙ is 2,3-dimethyl, X is N-2-methylpropionyl-N-ethylamino and R¹ for each compound corresponds to one row of table A

Table 106

Compounds of the formula IA, in which Rₙ is 2-chloro-6-fluoro, X is N-2-methylpropionyl-N-ethylamino and R¹ for each compound corresponds to one row of table A

Table 107

Compounds of the formula IA, in which Rₙ is 2-chloro-6-methyl, X is N-2-methylpropionyl-N-ethylamino and R¹ for each compound corresponds to one row of table A

Table 108

Compounds of the formula IA, in which Rₙ is 2-fluoro-6-methyl, X is N-2-methylpropionyl-N-ethylamino and R¹ for each compound corresponds to one row of table A

Table 109

Compounds of the formula IA, in which Rₙ is 2-chloro, X is methylsulfonyl and R¹ for each compound corresponds to one row of table A

Table 110

Compounds of the formula IA, in which Rₙ is 2-fluoro, X is methylsulfonyl and R¹ for each compound corresponds to one row of table A

Table 111

Compounds of the formula IA, in which Rₙ is 2-methyl, X is methylsulfonyl and R¹ for each compound corresponds to one row of table A

Table 112

Compounds of the formula IA, in which Rₙ is 2,6-dichloro, X is ethylsulfonyl and R¹ for each compound corresponds to one row of table A

Table 113

Compounds of the formula IA, in which Rₙ is 2,6-difluoro, X is ethylsulfonyl and R¹ for each compound corresponds to one row of table A

Table 114

Compounds of the formula IA, in which Rₙ is 2,6-dimethyl, X is methylsulfonyl and R¹ for each compound corresponds to one row of table A

Table 115

Compounds of the formula IA, in which Rₙ is 2-chloro-3-methyl, X is methylsulfonyl and R¹ for each compound corresponds to one row of table A

Table 116

Compounds of the formula IA, in which Rₙ is 2-fluoro-3-methyl, X is methylsulfonyl and R¹ for each compound corresponds to one row of table A

Table 117

Compounds of the formula IA, in which Rₙ is 2,3-dimethyl, X is methylsulfonyl and R¹ for each compound corresponds to one row of table A

Table 118

Compounds of the formula IA, in which Rₙ is 2-chloro-6-fluoro, X is methylsulfonyl and R¹ for each compound corresponds to one row of table A

Table 119

Compounds of the formula IA, in which Rₙ is 2-chloro-6-methyl, X is methylsulfonyl and R¹ for each compound corresponds to one row of table A

Table 120

Compounds of the formula IA, in which Rₙ is 2-fluoro-6-methyl, X is methylsulfonyl and R¹ for each compound corresponds to one row of table A

Table 121

Compounds of the formula IA, in which Rₙ is 2-chloro, X is ethylsulfonyl and R¹ for each compound corresponds to one row of table A

Table 122

Compounds of the formula IA, in which Rₙ is 2-fluoro, X is ethylsulfonyl and R¹ for each compound corresponds to one row of table A

Table 123

Compounds of the formula IA, in which Rₙ is 2-methyl, X is ethylsulfonyl and R¹ for each compound corresponds to one row of table A

Table 124

Compounds of the formula IA, in which Rₙ is 2,6-dichloro, X is ethylsulfonyl and R¹ for each compound corresponds to one row of table A

Table 125

Compounds of the formula IA, in which Rₙ is 2,6-difluoro, X is ethylsulfonyl and R¹ for each compound corresponds to one row of table A

Table 126

Compounds of the formula IA, in which Rₙ is 2,6-dimethyl, X is ethylsulfonyl and R¹ for each compound corresponds to one row of table A

Table 127

Compounds of the formula IA, in which Rₙ is 2-chloro-3-methyl, X is ethylsulfonyl and R¹ for each compound corresponds to one row of table A

Table 128

Compounds of the formula IA, in which Rₙ is 2-fluoro-3-methyl, X is ethylsulfonyl and R¹ for each compound corresponds to one row of table A

Table 129

Compounds of the formula IA, in which Rₙ is 2,3-dimethyl, X is ethylsulfonyl and R¹ for each compound corresponds to one row of table A

Table 130

Compounds of the formula IA, in which Rₙ is 2-chloro-6-fluoro, X is ethylsulfonyl and R¹ for each compound corresponds to one row of table A

Table 131

Compounds of the formula IA, in which Rₙ is 2-chloro-6-methyl, X is ethylsulfonyl and R¹ for each compound corresponds to one row of table A

Table 132

Compounds of the formula IA, in which Rₙ is 2-fluoro-6-methyl, X is ethylsulfonyl and R¹ for each compound corresponds to one row of table A

Table 133

Compounds of the formula IA, in which Rₙ is 2-chloro, X is 2-methylpropylsulfonyl and R¹ for each compound corresponds to one row of table A

Table 134

Compounds of the formula IA, in which Rₙ is 2-fluoro, X is 2-methylpropylsulfonyl and R¹ for each compound corresponds to one row of table A

Table 135

Compounds of the formula IA, in which Rₙ is 2-methyl, X is 2-methylpropylsulfonyl and R¹ for each compound corresponds to one row of table A

Table 136

Compounds of the formula IA, in which Rₙ is 2,6-dichloro, X is 2-methylpropylsulfonyl and R¹ for each compound corresponds to one row of table A

Table 137

Compounds of the formula IA, in which Rₙ is 2,6-difluoro, X is 2-methylpropylsulfonyl and R¹ for each compound corresponds to one row of table A

Table 138

Compounds of the formula IA, in which Rₙ is 2,6-dimethyl, X is 2-methylpropylsulfonyl and R¹ for each compound corresponds to one row of table A

Table 139

Compounds of the formula IA, in which Rₙ is 2-chloro-3-methyl, X is 2-methylpropylsulfonyl and R¹ for each compound corresponds to one row of table A

Table 140

Compounds of the formula IA, in which Rₙ is 2-fluoro-3-methyl, X is 2-methylpropylsulfonyl and R¹ for each compound corresponds to one row of table A

Table 141

Compounds of the formula IA, in which Rₙ is 2,3-dimethyl, X is 2-methylpropylsulfonyl and R¹ for each compound corresponds to one row of table A

Table 142

Compounds of the formula IA, in which Rₙ is 2-chloro-6-fluoro, X is 2-methylpropylsulfonyl and R¹ for each compound corresponds to one row of table A

Table 143

Compounds of the formula IA, in which Rₙ is 2-chloro-6-methyl, X is 2-methylpropylsulfonyl and R¹ for each compound corresponds to one row of table A

Table 144

Compounds of the formula IA, in which Rₙ is 2-fluoro-6-methyl, X is 2-methylpropylsulfonyl and R¹ for each compound corresponds to one row of table A

Table 145

Compounds of the formula IA, in which Rₙ is 2-chloro, X is methylsulfoxyl and R¹ for each compound corresponds to one row of table A

Table 146

Compounds of the formula IA, in which Rₙ is 2-fluoro, X is methylsulfoxyl and R¹ for each compound corresponds to one row of table A

Table 147

Compounds of the formula IA, in which Rₙ is 2-methyl, X is methylsulfoxyl and R¹ for each compound corresponds to one row of table A

Table 148

Compounds of the formula IA, in which Rₙ is 2,6-dichloro, X is methylsulfoxyl and R¹ for each compound corresponds to one row of table A

Table 149

Compounds of the formula IA, in which Rₙ is 2,6-difluoro, X is methylsulfoxyl and R¹ for each compound corresponds to one row of table A

Table 150

Compounds of the formula IA, in which Rₙ is 2,6-dimethyl, X is methylsulfoxyl and R¹ for each compound corresponds to one row of table A

Table 151

Compounds of the formula IA, in which Rₙ is 2-chloro-3-methyl, X is methylsulfoxyl and R¹ for each compound corresponds to one row of table A

Table 152

Compounds of the formula IA, in which Rₙ is 2-fluoro-3-methyl, X is methylsulfoxyl and R¹ for each compound corresponds to one row of table A

Table 153

Compounds of the formula IA, in which Rₙ is 2,3-dimethyl, X is methylsulfoxyl and R¹ for each compound corresponds to one row of table A

Table 154

Compounds of the formula IA, in which Rₙ is 2-chloro-6-fluoro, X is methylsulfoxyl and R¹ for each compound corresponds to one row of table A

Table 155

Compounds of the formula IA, in which Rₙ is 2-chloro-6-methyl, X is methylsulfoxyl and R¹ for each compound corresponds to one row of table A

Table 156

Compounds of the formula IA, in which Rₙ is 2-fluoro-6-methyl, X is methylsulfoxyl and R¹ for each compound corresponds to one row of table A

Table 157

Compounds of the formula IA, in which Rₙ is 2-chloro, X is ethylsulfoxyl and R¹ for each compound corresponds to one row of table A

Table 158

Compounds of the formula IA, in which Rₙ is 2-fluoro, X is ethylsulfoxyl and R¹ for each compound corresponds to one row of table A

Table 159

Compounds of the formula IA, in which Rₙ is 2-methyl, X is ethylsulfoxyl and R¹ for each compound corresponds to one row of table A

Table 160

Compounds of the formula IA, in which Rₙ is 2,6-dichloro, X is ethylsulfoxyl and R¹ for each compound corresponds to one row of table A

Table 161

Compounds of the formula IA, in which Rₙ is 2,6-difluoro, X is ethylsulfoxyl and R¹ for each compound corresponds to one row of table A

Table 162

Compounds of the formula IA, in which Rₙ is 2,6-dimethyl, X is ethylsulfoxyl and R¹ for each compound corresponds to one row of table A

Table 163

Compounds of the formula IA, in which Rₙ is 2-chloro-3-methyl, X is ethylsulfoxyl and R¹ for each compound corresponds to one row of table A

Table 164

Compounds of the formula IA, in which Rₙ is 2-fluoro-3-methyl, X is ethylsulfoxyl and R¹ for each compound corresponds to one row of table A

Table 165

Compounds of the formula IA, in which Rₙ is 2,3-dimethyl, X is ethylsulfoxyl and R¹ for each compound corresponds to one row of table A

Table 166

Compounds of the formula IA, in which Rₙ is 2-chloro-6-fluoro, X is ethylsulfoxyl and R¹ for each compound corresponds to one row of table A

Table 167

Compounds of the formula IA, in which Rₙ is 2-chloro-6-methyl, x is ethylsulfoxyl and R¹ for each compound corresponds to one row of table A

Table 168

Compounds of the formula IA, in which Rₙ is 2-fluoro-6-methyl, X is ethylsulfoxyl and R¹ for each compound corresponds to one row of table A

Table 169

Compounds of the formula IA, in which Rₙ is 2-chloro, X is 2-methylpropylsulfoxyl and R¹ for each compound corresponds to one row of table A

Table 170

Compounds of the formula IA, in which Rₙ is 2-fluoro, X is 2-methylpropylsulfoxyl and R¹ for each compound corresponds to one row of table A

Table 171

Compounds of the formula IA, in which Rₙ is 2-methyl, X is 2-methylpropylsulfoxyl and R¹ for each compound corresponds to one row of table A

Table 172

Compounds of the formula IA, in which Rₙ is 2,6-dichloro, X is 2-methylpropylsulfoxyl and R¹ for each compound corresponds to one row of table A

Table 173

Compounds of the formula IA, in which Rₙ is 2,6-difluoro, X is 2-methylpropylsulfoxyl and R¹ for each compound corresponds to one row of table A

Table 174

Compounds of the formula IA, in which Rₙ is 2,6-dimethyl, X is 2-methylpropylsulfoxyl and R¹ for each compound corresponds to one row of table A

Table 175

Compounds of the formula IA, in which Rₙ is 2-chloro-3-methyl, X is 2-methylpropylsulfoxyl and R¹ for each compound corresponds to one row of table A

Table 176

Compounds of the formula IA, in which Rₙ is 2-fluoro-3-methyl, X is 2-methylpropylsulfoxyl and R¹ for each compound corresponds to one row of table A

Table 177

Compounds of the formula IA, in which Rₙ is 2,3-dimethyl, X is 2-methylpropylsulfoxyl and R¹ for each compound corresponds to one row of table A

Table 178

Compounds of the formula IA, in which Rₙ is 2-chloro-6-fluoro, X is 2-methylpropylsulfoxyl and R¹ for each compound corresponds to one row of table A

Table 179

Compounds of the formula IA, in which Rₙ is 2-chloro-6-methyl, X is 2-methylpropylsulfoxyl and R¹ for each compound corresponds to one row of table A

Table 180

Compounds of the formula IA, in which Rₙ is 2-fluoro-6-methyl, X is 2-methylpropylsulfoxyl and R¹ for each compound corresponds to one row of table A

TABLE A

| No. | R¹ |
|---|---|
| A-1 | CH₃ |
| A-2 | CH₂CH₃ |
| A-3 | CH₂CH₂CH₃ |
| A-4 | CH(CH₃)₂ |
| A-5 | CH₂CH(CH₃)₂ |
| A-6 | (±)CH(CH₃)CH₂CH₃ |
| A-7 | (R)CH(CH₃)CH₂CH₃ |
| A-8 | (S)CH(CH₃)CH₂CH₃ |
| A-9 | (CH₂)₃CH₃ |
| A-10 | C(CH₃)₃ |
| A-11 | (CH₂)₄CH₃ |
| A-12 | CH(CH₂CH₃)₂ |
| A-13 | CH₂CH₂CH(CH₃)₂ |
| A-14 | (±)CH(CH₃)(CH₂)₂CH₃ |
| A-15 | (R)CH(CH₃)(CH₂)₂CH₃ |
| A-16 | (S)CH(CH₃)(CH₂)₂CH₃ |
| A-17 | (±)CH₂CH(CH₃)CH₂CH₃ |
| A-18 | (R CH₂CH(CH₃)CH₂CH₃ |
| A-19 | (S)CH₂CH(CH₃)CH₂CH₃ |
| A-20 | (±)CH(CH₃)CH(CH₃)₂ |
| A-21 | (R)CH(CH₃)CH(CH₃)₂ |
| A-22 | (S)CH(CH₃)CH(CH₃)₂ |
| A-23 | (CH₂)₅CH₃ |
| A-24 | (±,±)CH(CH₃)CH(CH₃)CH₂CH₃ |
| A-25 | (±,R)CH(CH₃)CH(CH₃)CH₂CH₃ |
| A-26 | (±,S)CH(CH₃)CH(CH₃)CH₂CH₃ |
| A-27 | (±)CH₂CH(CH₃)CF₃ |
| A-28 | (R)CH₂CH(CH₃)CF₃ |
| A-29 | (S)CH₂CH(CH₃)CF₃ |
| A-30 | (±)CH₂CH(CF₃)CH₂CH₃ |
| A-31 | (R)CH₂CH(CF₃)CH₂CH₃ |
| A-32 | (S)CH₂CH(CF₃)CH₂CH₃ |
| A-33 | (±,±)CH(CH₃)CH(CH₃)CF₃ |
| A-34 | (±,R)CH(CH₃)CH(CH₃)CF₃ |
| A-35 | (±,S)CH(CH₃)CH(CH₃)CF₃ |
| A-36 | (±,±)CH(CH₃)CH(CF₃)CH₂CH₃ |
| A-37 | (±,R)CH(CH₃)CH(CF₃)CH₂CH₃ |
| A-38 | (±,S)CH(CH₃)CH(CF₃)CH₂CH₃ |
| A-39 | CF₃ |
| A-40 | CF₂CF₃ |
| A-41 | CF₂CF₂CF₃ |
| A-42 | c-C₃H₅ |
| A-43 | (1-CH₃)-c-C₃H₄ |
| A-44 | c-C₅H₉ |
| A-45 | c-C₆H₁₁ |
| A-46 | (4-CH₃)-c-C₆H₁₀ |
| A-47 | CH₂C(CH₃)═CH₂ |
| A-48 | CH₂CH₂C(CH₃)═CH₂ |
| A-49 | CH₂—C(CH₃)₃ |
| A-50 | CH₂—Si(CH₃)₃ |
| A-51 | n-C₆H₁₃ |
| A-52 | (CH₂)₃—CH(CH₃)₂ |
| A-53 | (CH₂)₂—CH(CH₃)—C₂H₅ |
| A-54 | CH₂—CH(CH₃)-n-C₃H₇ |
| A-55 | CH(CH₃)-n-C₄H₉ |
| A-56 | CH₂—CH(C₂H₅)₂ |
| A-57 | CH(C₂H₅)-n-C₃H₇ |
| A-58 | CH₂-c-C₅H₉ |
| A-59 | CH₂—CH(CH₃)—CH(CH₃)₂ |
| A-60 | CH(CH₃)—CH₂CH(CH₃)₂ |
| A-61 | CH(CH₃)—CH(CH₃)—C₂H₅ |
| A-62 | CH(CH₃)—C(CH₃)₃ |
| A-63 | (CH₂)₂—C(CH₃)₃ |
| A-64 | CH₂—C(CH₃)₂—C₂H₅ |
| A-65 | 2-CH₃-c-C₅H₈ |
| A-66 | 3-CH₃-c-C₅H₈ |
| A-67 | C(CH₃)₂-n-C₃H₇ |
| A-68 | (CH₂)₆—CH₃ |
| A-69 | (CH₂)₄—CH(CH₃)₂ |
| A-70 | (CH₂)₃—CH(CH₃)—C₂H₅ |
| A-71 | (CH₂)₂—CH(CH₃)-n-C₃H₇ |
| A-72 | CH₂—CH(CH₃)-n-C₄H₉ |
| A-73 | CH(CH₃)-n-C₅H₁₁ |
| A-74 | (CH₂)₃C(CH₃)₃ |
| A-75 | (CH₂)₂CH(CH₃)—CH(CH₃)₂ |
| A-76 | (CH₂)CH(CH₃)—CH₂CH(CH₃)₂ |
| A-77 | CH(CH₃)(CH₂)₂—CH(CH₃)₂ |
| A-78 | (CH₂)₂C(CH₃)₂C₂H₅ |
| A-79 | CH₂CH(CH₃)CH(CH₃)C₂H₅ |
| A-80 | CH(CH₃)CH₂CH(CH₃)C₂H₅ |
| A-81 | CH₂C(CH₃)₂-n-C₃H₇ |
| A-82 | CH(CH₃)CH(CH₃)-n-C₃H₇ |
| A-83 | C(CH₃)₂-n-C₄H₉ |
| A-84 | (CH₂)₂CH(C₂H₅)₂ |
| A-85 | CH₂CH(C₂H₅)-n-C₃H₇ |
| A-86 | CH(C₂H₅)-n-C₄H₉ |
| A-87 | CH₂CH(CH₃)C(CH₃)₃ |
| A-88 | CH(CH₃)CH₂C(CH₃)₃ |
| A-89 | CH₂C(CH₃)₂CH(CH₃)₂ |
| A-90 | CH₂CH(C₂H₅)CH(CH₃)₂ |
| A-91 | CH(CH₃)CH(CH₃)CH(CH₃)₂ |
| A-92 | C(CH₃)₂CH₂CH(CH₃)₂ |
| A-93 | CH(C₂H₅)CH₂CH(CH₃)₂ |
| A-94 | CH(CH₃)C(CH₃)₂C₂H₅ |
| A-95 | CH(CH₃)CH(C₂H₅)₂ |
| A-96 | C(CH₃)₂CH(CH₃)C₂H₅ |
| A-97 | CH(C₂H₅)CH(CH₃)C₂H₅ |
| A-98 | C(CH₃)(C₂H₅)-n-C₃H₇ |
| A-99 | CH(n-C₃H₇)₂ |
| A-100 | CH(n-C₃H₇)CH(CH₃)₂ |
| A-101 | C(CH₃)₂C(CH₃)₃ |
| A-102 | C(CH₃)(C₂H₅)—CH(CH₃)₂ |
| A-103 | C(C₂H₅)₃ |
| A-104 | (3-CH₃)-c-C₆H₁₀ |
| A-105 | (2-CH₃)-c-C₆H₁₀ |
| A-106 | n-C₈H₁₇ |
| A-107 | CH₂C(═NO—CH₃)CH₃ |
| A-108 | CH₂C(═NO—C₂H₅)CH₃ |
| A-109 | CH₂C(═NO-n-C₃H₇)CH₃ |
| A-110 | CH₂C(═NO-i-C₃H₇)CH₃ |
| A-111 | CH(CH₃)C(═NOCH₃)CH₃ |
| A-112 | CH(CH₃)C(═NOC₂H₅)CH₃ |
| A-113 | CH(CH₃)C(═NO-n-C₃H₇)CH₃ |
| A-114 | CH(CH₃)C(═NO-i-C₃H₇)CH₃ |
| A-115 | C(═NOCH₃)C(═NOCH₃)CH₃ |
| A-116 | C(═NOCH₃)C(═NOC₂H₅)CH₃ |
| A-117 | C(═NOCH₃)C(═NO-n-C₃H₇)CH₃ |
| A-118 | C(═NOCH₃)C(═NO-i-C₃H₇)CH₃ |
| A-119 | C(═NOC₂H₅)C(═NOCH₃)CH₃ |
| A-120 | C(═NOC₂H₅)C(═NOC₂H₅)CH₃ |
| A-121 | C(═NOC₂H₅)C(═NO-n-C₃H₇)CH₃ |
| A-122 | C(═NOC₂H₅)C(═NO-i-C₃H₇)CH₃ |
| A-123 | CH₂C(═NO—CH₃)C₂H₅ |
| A-124 | CH₂C(═NO—C₂H₅)C₂H₅ |
| A-125 | CH₂C(═NO-n-C₃H₇)C₂H₅ |
| A-126 | CH₂C(═NO-i-C₃H₇)C₂H₅ |
| A-127 | CH(CH₃)C(═NOCH₃)C₂H₅ |
| A-128 | CH(CH₃)C(═NOC₂H₅)C₂H₅ |
| A-129 | CH(CH₃)C(═NO-n-C₃H₇)C₂H₅ |
| A-130 | CH(CH₃)C(═NO-n-C₃H₇)C₂H₅ |
| A-131 | C(═NOCH₃)C(═NOCH₃)C₂H₅ |
| A-132 | C(═NOCH₃)C(═NOC₂H₅)C₂H₅ |
| A-133 | C(═NOCH₃)C(═NO-n-C₃H₇)C₂H₅ |
| A-134 | C(═NOCH₃)C(═NO-i-C₃H₇)C₂H₅ |
| A-135 | C(═NOC₂H₅)C(═NOCH₃)C₂H₅ |
| A-136 | C(═NOC₂H₅)C(═NOC₂H₅)C₂H₅ |
| A-137 | C(═NOC₂H₅)C(═NO-n-C₃H₇)C₂H₅ |
| A-138 | C(═NOC₂H₅)C(═NO-i-C₃H₇)C₂H₅ |
| A-139 | CH═CH—CH₂CH₃ |
| A-140 | CH₂—CH═CH—CH₃ |
| A-141 | CH₂—CH₂—CH═CH₂ |
| A-142 | C(CH₃)₂CH₂CH₃ |
| A-143 | CH═C(CH₃)₂ |
| A-144 | C(═CH₂)—CH₂CH₃ |
| A-145 | C(CH₃)═CH—CH₃ |
| A-146 | CH(CH₃)CH═CH₂ |
| A-147 | CH═CH-n-C₃H₇ |
| A-148 | CH₂—CH═CH—C₂H₅ |
| A-149 | (CH₂)₂—CH═CH—CH₃ |
| A-150 | (CH₂)₃—CH═CH₂ |
| A-151 | CH═CH—CH(CH₃)₂ |
| A-152 | CH₂—CH═C(CH₃)₂ |
| A-153 | (CH₂)₂—C(CH₃)═CH₂ |
| A-154 | CH═C(CH₃)—C₂H₅ |
| A-155 | CH₂—C(═CH₂)—C₂H₅ |
| A-156 | CH₂—C(CH₃)═CH—CH₃ |
| A-157 | CH₂—CH(CH₃)—CH═CH₂ |
| A-158 | C(═CH₂)—CH₂—CH₂—CH₃ |
| A-159 | C(CH₃)═CH—CH₂—CH₃ |
| A-160 | CH(CH₃)—CH═CH—CH₃ |
| A-161 | CH(CH₃)—CH₂—CH═CH₂ |
| A-162 | C(═CH₂)CH(CH₃)₂ |
| A-163 | C(CH₃)═C(CH₃)₂ |
| A-164 | CH(CH₃)—C(═CH₂)—CH₃ |
| A-165 | C(CH₃)₂—CH═CH₂ |
| A-166 | C(C₂H₅)═CH—CH₃ |
| A-167 | CH(C₂H₅)—CH═CH₂ |
| A-168 | CH═CH—CH₂—CH₂—CH₂—CH₃ |
| A-169 | CH₂—CH═CH—CH₂—CH₂—CH₃ |
| A-170 | CH₂—CH₂—CH═CH—CH₂—CH₃ |
| A-171 | CH₂—CH₂—CH₂—CH═CH—CH₃ |
| A-172 | CH₂—CH₂—CH₂—CH₂—CH═CH₂ |
| A-173 | CH═CH—CH₂—CH(CH₃)CH₃ |
| A-174 | CH₂—CH═CH—CH(CH₃)CH₃ |
| A-175 | CH₂—CH₂—CH═C(CH₃)CH₃ |
| A-176 | CH₂—CH₂—CH₂—C(CH₃)═CH₂ |
| A-177 | CH═CH—CH(CH₃)—CH₂—CH₃ |
| A-178 | CH₂—CH═C(CH₃)—CH₂—CH₃ |
| A-179 | CH₂—CH₂—C(═CH₂)—CH₂—CH₃ |
| A-180 | CH₂—CH₂—C(CH₃)═CH—CH₃ |
| A-181 | CH₂—CH₂—CH(CH₃)—CH═CH₂ |
| A-182 | CH═C(CH₃)—CH₂—CH₂—CH₃ |
| A-183 | CH₂—C(═CH₂)—CH₂—CH₂—CH₃ |
| A-184 | CH₂—C(CH₃)═CH—CH₂—CH₃ |
| A-185 | CH₂—CH(CH₃)—CH═CH—CH₃ |
| A-186 | CH₂—CH(CH₃)—CH₂—CH═CH₂ |
| A-187 | C(═CH₂)—CH₂—CH₂—CH₂—CH₃ |
| A-188 | C(CH₃)═CH—CH₂—CH₂—CH₃ |
| A-189 | CH(CH₃)—CH═CH—CH₂—CH₃ |
| A-190 | CH(CH₃)—CH₂—CH═CH—CH₃ |
| A-191 | CH(CH₃)—CH₂—CH₂—CH═CH₂ |
| A-192 | CH═CH—C(CH₃)₃ |
| A-193 | CH═C(CH₃)—CH(CH₃)—CH₃ |
| A-194 | CH₂—C(═CH₂)—CH(CH₃)—CH₃ |
| A-195 | CH₂—C(CH₃)═C(CH₃)—CH₃ |
| A-196 | CH₂—CH(CH₃)—C(═CH₂)—CH₃ |
| A-197 | C(═CH₂)—CH₂—CH(CH₃)—CH₃ |
| A-198 | C(CH₃)═CH—CH(CH₃)—CH₃ |
| A-199 | CH(CH₃)—CH═C(CH₃)—CH₃ |
| A-200 | CH(CH₃)—CH₂—C(═CH₂)—CH₃ |
| A-201 | CH═C(CH₂—CH₃)—CH₂—CH₃ |
| A-202 | CH₂—C(═CH—CH₃)—CH₂—CH₃ |
| A-203 | CH₂—CH(CH═CH₂)—CH₂—CH₃ |
| A-204 | C(═CH—CH₃)—CH₂—CH₂—CH₃ |
| A-205 | CH(CH═CH₂)—CH₂—CH₂—CH₃ |
| A-206 | C(CH₂—CH₃)═CH—CH₂—CH₃ |
| A-207 | CH(CH₂—CH₃)—CH═CH—CH₃ |
| A-208 | CH(CH₂—CH₃)—CH₂—CH═CH₂ |
| A-209 | CH₂—C(CH₃)₂—CH═CH₂ |
| A-210 | C(═CH₂)—CH(CH₃)—CH₂—CH₃ |
| A-211 | C(CH₃)═C(CH₃)—CH₂—CH₃ |
| A-212 | CH(CH₃)—C(═CH₂)—CH₂—CH₃ |
| A-213 | CH(CH₃)—C(CH₃)═CH—CH₃ |
| A-214 | CH(CH₃)—CH(CH₃)—CH═CH₂ |
| A-215 | C(CH₃)₂—CH═CH—CH₃ |
| A-216 | C(CH₃)₂—CH₂—CH═CH₂ |
| A-217 | C(═CH₂)—C(CH₃)₃ |
| A-218 | C(═CH—CH₃)—CH(CH₃)—CH₃ |
| A-219 | CH(CH═CH₂)—CH(CH₃)—CH₃ |
| A-220 | C(CH₂—CH₃)═C(CH₃)—CH₃ |
| A-221 | CH(CH₂—CH₃)—C(═CH₂)—CH₃ |
| A-222 | C(CH₃)₂—C(═CH₂)—CH₃ |
| A-223 | C(CH₃)(CH═CH₂)—CH₂—CH₃ |
| A-224 | C(CH₃)(CH₂CH₃)—CH₂—CH₂—CH₃ |
| A-225 | CH(CH₂CH₃)—CH(CH₃)—CH₂—CH₃ |
| A-226 | CH(CH₂CH₃)—CH₂—CH(CH₃)—CH₃ |
| A-227 | C(CH₃)₂—C(CH₃)₃ |
| A-228 | C(CH₂—CH₃)—C(CH₃)₃ |
| A-229 | C(CH₃)(CH₂—CH₃)—CH(CH₃)₂ |
| A-230 | CH(CH(CH₃)₂)—CH(CH₃)₂ |
| A-231 | CH═CH—CH₂—CH₂—CH₂—CH₂—CH₃ |
| A-232 | CH₂—CH═CH—CH₂—CH₂—CH₂—CH₃ |
| A-233 | CH₂—CH₂—CH═CH—CH₂—CH₂—CH₃ |
| A-234 | CH₂—CH₂—CH₂—CH═CH—CH₂—CH₃ |
| A-235 | CH₂—CH₂—CH₂—CH₂—CH═CH—CH₃ |
| A-236 | CH₂—CH₂—CH₂—CH₂—CH₂—CH═CH₂ |
| A-237 | CH═CH—CH₂—CH₂—CH(CH₃)—CH₃ |
| A-238 | CH₂—CH═CH—CH₂—CH(CH₃)—CH₃ |
| A-239 | CH₂—CH₂—CH═CH—CH(CH₃)—CH₃ |
| A-240 | CH₂—CH₂—CH₂—CH═C(CH₃)—CH₃ |
| A-241 | CH₂—CH₂—CH₂—CH₂—C(═CH₂)—CH₃ |
| A-242 | CH═CH—CH₂—CH(CH₃)—CH₂—CH₃ |
| A-243 | CH₂—CH═CH—CH(CH₃)—CH₂—CH₃ |
| A-244 | CH₂—CH₂—CH═C(CH₃)—CH₂—CH₃ |
| A-245 | CH₂—CH₂—CH₂—C(═CH₂)—CH₂—CH₃ |
| A-246 | CH₂—CH₂—CH₂—C(CH₃)═CH—CH₃ |
| A-247 | CH₂—CH₂—CH₂—CH(CH₃)—CH═CH₂ |
| A-248 | CH═CH—CH(CH₃)—CH₂—CH₂—CH₃ |
| A-249 | CH₂—CH═C(CH₃)—CH₂—CH₂—CH₃ |
| A-250 | CH₂—CH₂—C(═CH₂)—CH₂—CH₂—CH₃ |
| A-251 | CH₂—CH₂—C(CH₃)═CH—CH₂—CH₃ |
| A-252 | CH₂—CH₂—CH(CH₃)—CH═CH—CH₃ |
| A-253 | CH₂—CH₂—CH(CH₃)—CH₂—CH═CH₂ |
| A-254 | CH═C(CH₃)—CH₂—CH₂—CH₂—CH₃ |
| A-255 | CH₂—C(═CH₂)—CH₂—CH₂—CH₂—CH₃ |
| A-256 | CH₂—C(CH₃)═CH—CH₂—CH₂—CH₃ |
| A-257 | CH₂—CH(CH₃)—CH═CH—CH₂—CH₃ |
| A-258 | CH₂—CH(CH₃)—CH₂—CH═CH—CH₃ |
| A-259 | CH₂—CH(CH₃)—CH₂—CH₂—CH═CH₂ |
| A-260 | C(═CH₂)—CH₂—CH₂—CH₂—CH₂—CH₃ |
| A-261 | C(CH₃)═CH—CH₂—CH₂—CH₂—CH₃ |
| A-262 | CH(CH₃)—CH═CH—CH₂—CH₂—CH₃ |
| A-263 | CH(CH₃)—CH₂—CH═CH—CH₂—CH₃ |
| A-264 | CH(CH₃)—CH₂—CH₂—CH═CH—CH₃ |
| A-265 | CH(CH₃)—CH₂—CH₂—CH₂—CH═CH₂ |
| A-266 | CH═CH—CH₂—C(CH₃)₃ |
| A-267 | CH₂—CH═CH—C(CH₃)₃ |
| A-268 | CH═CH—CH(CH₃)—CH(CH₃)₂ |
| A-269 | CH₂—CH═C(CH₃)—CH(CH₃)₂ |
| A-270 | CH₂—CH₂—C(═CH₂)—CH(CH₃)₂ |
| A-271 | CH₂—CH₂—C(CH₃)═C(CH₃)₂ |
| A-272 | CH₂—CH₂—CH(CH₃)—C(═CH₂)—CH₃ |
| A-273 | CH═C(CH₃)—CH₂—CH(CH₃)₂ |
| A-274 | CH₂—C(═CH₂)—CH₂—CH(CH₃)₂ |
| A-275 | CH₂—C(CH₃)═CH—CH(CH₃)₂ |
| A-276 | CH₂—CH(CH₃)—CH═C(CH₃)₂ |
| A-277 | CH₂—CH(CH₃)—CH₂—C(═CH₂)—CH₃ |
| A-278 | C(═CH₂)—CH₂—CH₂—CH(CH₃)₂ |
| A-279 | C(CH₃)═CH—CH₂—CH(CH₃)₂ |
| A-280 | CH(CH₃)—CH═CH—CH(CH₃)₂ |
| A-281 | CH(CH₃)—CH₂—CH═C(CH₃)₂ |
| A-282 | CH(CH₃)—CH₂—CH₂—C(═CH₂)—CH₃ |
| A-283 | CH═CH—C(CH₃)₂—CH₂—CH₃ |
| A-284 | CH₂—CH₂—C(CH₃)₂—CH═CH₂ |
| A-285 | CH═C(CH₃)—CH(CH₃)—CH₂—CH₃ |
| A-286 | CH₂—C(═CH₂)—CH(CH₃)—CH₂—CH₃ |
| A-287 | CH₂—C(CH₃)═C(CH₃)—CH₂—CH₃ |
| A-288 | CH₂—CH(CH₃)—C(═CH₂)—CH₂—CH₃ |
| A-289 | CH₂—CH(CH₃)—C(CH₃)═CH—CH₃ |
| A-290 | CH₂—CH(CH₃)—CH(CH₃)—CH═CH₂ |
| A-291 | C(═CH₂)—CH₂—CH(CH₃)—CH₂—CH₃ |
| A-292 | C(CH₃)═CH—CH(CH₃)—CH₂—CH₃ |
| A-293 | CH(CH₃)—CH═C(CH₃)—CH₂—CH₃ |
| A-294 | CH(CH₃)—CH₂—C(═CH₂)—CH₂—CH₃ |
| A-295 | CH(CH₃)—CH₂—C(CH₃)═CH—CH₃ |
| A-296 | CH(CH₃)—CH₂—CH(CH₃)—CH═CH₂ |
| A-297 | CH₂—C(CH₃)₂—CH═CH—CH₃ |
| A-298 | CH₂—C(CH₃)₂—CH₂—CH═CH₂ |
| A-299 | C(═CH₂)—CH(CH₃)—CH₂—CH₂—CH₃ |
| A-300 | C(CH₃)═C(CH₃)—CH₂—CH₂—CH₃ |
| A-301 | CH(CH₃)—C(═CH₂)—CH₂—CH₂—CH₃ |
| A-302 | CH(CH₃)—C(CH₃)═CH—CH₂—CH₃ |
| A-303 | CH(CH₃)—CH(CH₃)—CH═CH—CH₃ |
| A-304 | CH(CH₃)—CH(CH₃)—CH₂—CH═CH₂ |
| A-305 | C(CH₃)₂—CH═CH—CH₂—CH₃ |
| A-306 | C(CH₃)₂—CH₂—CH═CH—CH₃ |
| A-307 | C(CH₃)₂—CH₂—CH₂—CH═CH₂ |
| A-308 | CH═CH—CH(CH₂—CH₃)—CH₂—CH₃ |
| A-309 | CH₂—CH═C(CH₂—CH₃)—CH₂—CH₃ |
| A-310 | CH₂—CH₂—C(═CH—CH₃)—CH₂—CH₃ |
| A-311 | CH₂—CH₂—CH(CH═CH₂)—CH₂—CH₃ |
| A-312 | CH═C(CH₂—CH₃)—CH₂—CH₂—CH₃ |
| A-313 | CH₂—C(═CH—CH₃)—CH₂—CH₂—CH₃ |
| A-314 | CH₂—CH(CH═CH₂)—CH₂—CH₂—CH₃ |
| A-315 | CH₂—C(CH₂—CH₃)═CH—CH₂—CH₃ |
| A-316 | CH₂—CH(CH₂—CH₃)—CH═CH—CH₃ |
| A-317 | CH₂—CH(CH₂—CH₃)—CH—CH═CH₂ |
| A-318 | C(═CH—CH₃)—CH₂—CH₂—CH₂—CH₃ |
| A-319 | CH(CH═CH₂)—CH₂—CH₂—CH₂—CH₃ |
| A-320 | C(CH₂—CH₃)═CH—CH₂—CH₂—CH₃ |
| A-321 | CH(CH₂—CH₃)—CH═CH—CH₂—CH₃ |
| A-322 | CH(CH₂—CH₃)—CH₂—CH═CH—CH₃ |
| A-323 | CH(CH₂—CH₃)—CH₂—CH₂—CH═CH₂ |
| A-324 | C(═CH—CH₂—CH₃)—CH₂—CH₂—CH₃ |
| A-325 | C(CH═CH—CH₃)—CH₂—CH₂—CH₃ |
| A-326 | C(CH₂—CH═CH₂)—CH₂—CH₂—CH₃ |
| A-327 | CH═C(CH₃)—C(CH₃)₃ |
| A-328 | CH₂—C(═CH₂)—C(CH₃)₃ |
| A-329 | CH₂—C(CH₃)₂—CH(═CH₂)—CH₃ |
| A-330 | C(═CH₂)—CH(CH₃)—CH(CH₃)—CH₃ |
| A-331 | C(CH₃)═C(CH₃)—CH(CH₃)—CH₃ |
| A-332 | CH(CH₃)—C(═CH₂)—CH(CH₃)—CH₃ |
| A-333 | CH(CH₃)—C(CH₃)═C(CH₃)—CH₃ |
| A-334 | CH(CH₃)—CH(CH₃)—C(═CH₂)—CH₃ |
| A-335 | C(CH₃)₂—CH═C(CH₃)—CH₃ |
| A-336 | C(CH₃)₂—CH₂—C(═CH₂)—CH₃ |
| A-337 | C(CH₃)₂—C(═CH₂)—CH₂—CH₃ |
| A-338 | C(CH₃)₂—C(CH₃)═CH—CH₃ |
| A-339 | C(CH₃)₂—CH(CH₃)CH═CH₂ |
| A-340 | CH(CH₂—CH₃)—CH₂—CH(CH₃)—CH₃ |
| A-341 | CH(CH₂—CH₃)—CH(CH₃)—CH₂—CH₃ |
| A-342 | C(CH₃)(CH₂—CH₃)—CH₂—CH₂—CH₃ |
| A-343 | CH(i-C₃H₇)—CH₂—CH₂—CH₃ |
| A-344 | CH═C(CH₂—CH₃)—CH(CH₃)—CH₃ |
| A-345 | CH₂—C(═CH—CH₃)—CH(CH₃)—CH₃ |
| A-346 | CH₂—CH(CH═CH₂)—CH(CH₃)—CH₃ |
| A-347 | CH₂—C(CH₂—CH₃)═C(CH₃)—CH₃ |
| A-348 | CH₂—CH(CH₂—CH₃)—C(═CH₂)—CH₃ |
| A-349 | CH₂—C(CH₃)(CH═CH₂)—CH₂—CH₃ |
| A-350 | C(═CH₂)—CH(CH₂—CH₃)—CH₂—CH₃ |
| A-351 | C(CH₃)═C(CH₂—CH₃)—CH₂—CH₃ |
| A-352 | CH(CH₃)—C(═CH—CH₃)—CH₂—CH₃ |
| A-353 | CH(CH₃)—CH(CH═CH₂)—CH₂—CH₃ |
| A-354 | CH═C(CH₂—CH₃)—CH(CH₃)—CH₃ |
| A-355 | CH₂—C(═CH—CH₃)—CH(CH₃)—CH₃ |
| A-356 | CH₂—CH(CH═CH₂)—CH(CH₃)—CH₃ |
| A-357 | CH₂—C(CH₂—CH₃)═C(CH₃)—CH₃ |
| A-358 | CH₂—CH(CH₂—CH₃)—C(═CH₂)—CH₃ |
| A-359 | C(═CH—CH₃)—CH₂—CH(CH₃)—CH₃ |
| A-360 | CH(CH═CH₂)—CH₂—CH(CH₃)—CH₃ |
| A-361 | C(CH₂—CH₃)═CH—CH(CH₃)—CH₃ |
| A-362 | CH(CH₂—CH₃)CH═C(CH₃)—CH₃ |
| A-363 | CH(CH₂—CH₃)CH₂—C(═CH₂)—CH₃ |
| A-364 | C(═CH—CH₃)CH(CH₃)—CH₂—CH₃ |
| A-365 | CH(CH═CH₂)CH(CH₃)—CH₂—CH₃ |
| A-366 | C(CH₂—CH₃)═C(CH₃)—CH₂—CH₃ |
| A-367 | CH(CH₂—CH₃)—C(═CH₂)—CH₂—CH₃ |
| A-368 | CH(CH₂—CH₃)—C(CH₃)═CH—CH₃ |
| A-369 | CH(CH₂—CH₃)—CH(CH₃)—CH═CH₂ |
| A-370 | C(CH₃)(CH═CH₂)—CH₂—CH₂—CH₃ |
| A-371 | C(CH₃)(CH₂—CH₃)—CH═CH—CH₃ |
| A-372 | C(CH₃)(CH₂—CH₃)—CH₂—CH═CH₂ |
| A-373 | C[═C(CH₃)—CH₃]—CH₂—CH₂—CH₃ |
| A-374 | CH[C(═CH₂)—CH₃]—CH₂—CH₂—CH₃ |
| A-375 | C(i-C₃H₇)═CH—CH₂—CH₃ |
| A-376 | CH(i-C₃H₇)—CH═CH—CH₃ |
| A-377 | CH(i-C₃H₇)—CH₂—CH═CH₂ |
| A-378 | C(═CH—CH₃)—C(CH₃)₃ |
| A-379 | CH(CH═CH₂)—C(CH₃)₃ |
| A-380 | C(CH₃)(CH═CH₂)CH(CH₃)—CH₃ |
| A-381 | C(CH₃)(CH₂—CH₃)C(═CH₂)—CH₃ |
| A-382 | 2-CH₃-cyclohex-1-enyl |
| A-383 | [2-(═CH₂)]-c-C₆H₉ |
| A-384 | 2-CH₃-cyclohex-2-enyl |
| A-385 | 2-CH₃-cyclohex-3-enyl |
| A-386 | 2-CH₃-cyclohex-4-enyl |
| A-387 | 2-CH₃-cyclohex-5-enyl |
| A-388 | 2-CH₃-cyclohex-6-enyl |
| A-389 | 3-CH₃-cyclohex-1-enyl |
| A-390 | 3-CH₃-cyclohex-2-enyl |
| A-391 | [3-(═CH₂)]-c-C₆H₉ |
| A-392 | 3-CH₃-cyclohex-3-enyl |
| A-393 | 3-CH₃-cyclohex-4-enyl |
| A-394 | 3-CH₃-cyclohex-5-enyl |
| A-395 | 3-CH₃-cyclohex-6-enyl |
| A-396 | 4-CH₃-cyclohex-1-enyl |
| A-397 | 4-CH₃-cyclohex-2-enyl |
| A-398 | 4-CH₃-cyclohex-3-enyl |
| A-399 | [4-(═CH₂)]-c-C₆H₉ |

The compounds I are suitable as fungicides. They have excellent activity against a broad spectrum of phytopathogenic fungi, in particular from the class of the Ascomycetes, Deuteromycetes, Oomycetes and Basidiomycetes. Some of them act systemically and can be employed in crop protection as foliar- and soil-acting fungicides.

They are especially important for controlling a large number of fungi in a variety of crop plants such as wheat, rye, barley, oats, rice, maize, grass, bananas, cotton, soybean, coffee, sugar cane, grapevines, fruit species, ornamentals and vegetable species such as cucumbers, beans, tomatoes, potatoes and cucurbits, and also in the seeds of these plants.

Specifically, they are suitable for controlling the following plant diseases:
Alternaria species in vegetables and fruit,
Bipolaris and Drechslera species in cereals, rice and lawns,
Blumeria graminis (powdery mildew) in cereals,
Botrytis cinerea (gray mold) in strawberries, vegetables, ornamentals and grapevines,
Erysiphe cichoracearum and Sphaerotheca fuliginea in cucurbits,
Fusarium and Verticillium species in a variety of plants,
Mycosphaerella species in cereals, bananas and groundnuts,
Phytophthora infestans in potatoes and tomatoes,
Plasmopara viticola in grapevines,
Podosphaera leucotricha in apples,
Pseudocercosporella herpotrichoides in wheat and barley, Pseudoperonospora species in hops and cucumbers,
Puccinia species in cereals,
Pyricularia oryzae in rice,
Rhizoctonia species in cotton, rice and lawns,
Septoria tritici and Stagonospora nodorum in wheat,
Uncinula necator in grapevines,
Ustilago species in cereals and sugar cane, and
Venturia species (scab) in apples and pears.

The compounds I are also suitable for controlling harmful fungi such as Paecilomyces variotii in the protection of materials (for example wood, paper, paint dispersions, fibers or tissues) and in the protection of stored products.

The compounds I are employed by treating the fungi or the plants, seeds, materials or the soil to be protected against fungal attack with a fungicidally effective amount of the active compounds. The application can be carried out before or after the infection of the materials, plants or seeds by the fungi.

The fungicidal compositions generally comprise from 0.1 to 95, preferably from 0.5 to 90% by weight of active compound.

For use in crop protection, the application rates are, depending on the kind of effect desired, from 0.01 to 2.0 kg of active compound per ha.

The treatment of seeds generally requires active compound rates of from 0.001 to 0.1 g, preferably from 0.01 to 0.05 g, per kilogram of seed.

For use in the protection of materials or stored products, the active compound application rate depends on the kind of application area and effect desired. Customary application rates in the protection of materials are, for example, from 0.001 g to 2 kg, preferably from 0.005 g to 1 kg, of active compound per cubic meter of treated material.

The compounds I can be converted into the customary formulations, e.g. solutions, emulsions, suspensions, dusts, powders, pastes and granules. The use form depends on the specific intended use; in any case, it should ensure fine and uniform distribution of the compound according to the invention.

The formulations are prepared in a known manner, e.g. by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle solvent mixtures may also be used,
carriers such as ground natural minerals (e.g. kaolins, clays, talc, chalk) and ground synthetic minerals (e.g. finely divided silica, silicates); emulsifiers such as nonionic and anionic emulsifiers (e.g. polyoxyethylene fatty alcohol ethers, alkylsulfonates and arylsulfonates), and dispersants such as lignosulfite waste liquors and methylcellulose.

Suitable surfactants which can be used are the alkali metal, alkaline earth metal and ammonium salts of lignosulfonic acid, naphthalenesulfonic acid, phenolsulfonic acid, and dibutylnaphthalenesulfonic acid, alkylarylsulfonates, alkyl sulfates, alkylsulfonates, fatty alcohol sulfates, fatty acids and sulfated fatty alcohol glycol ethers, furthermore condensation products of sulfonated naphthalene and naphthalene derivatives with formaldehyde, condensation products of naphthalene or of naphthalene sulfonic acid with phenol and formaldehyde, polyoxyethylene octylphenol ethers, ethoxylated isooctylphenol, octylphenol and nonylphenol, alkylphenol polyglycol ethers, tributylphenyl polyglycol ethers, tristerylphenyl polyglycol ethers, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose are suitable.

Suitable for preparing directly sprayable solutions, emulsions, pastes or oil dispersions are mineral oil fractions having medium to high boiling points, such as kerosene or diesel fuel, furthermore coal-tar oils and oils of plant or animal origin, aliphatic, cyclic and aromatic hydrocarbons, for example, toluene, xylene, paraffin, tetrahydronaphthalene, alkylated naphthalenes or derivatives thereof, methanol, ethanol, propanol, butanol, cyclohexanol, cyclohexanone, isophorone, strongly polar solvents, for example dimethyl sulfoxide, N-methylpyrrolidone, or water.

Powders, compositions for broadcasting and dusts can be prepared by mixing or joint grinding of the active substances with a solid carrier.

Granules, for example coated granules, impregnated granules and homogenous granules, can be prepared by binding the active compounds to solid carriers. Solid carriers are, for example, mineral earths, such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as ammonium sulfate, ammonium phosphate, ammonium nitrate, ureas and products of plant origin, such as cereal meal, tree bark meal, wood meal and nutshell meal, cellulose powders and other solid carriers.

The formulations generally comprise from 0.01 to 95% by weight, preferably from 0.1 to 90% by weight, of the active compound. The active compounds are employed in a purity of from 90% to 100%, preferably from 95% to 100% (according to the NMR spectrum).

Examples of formulations are: 1. Products for dilution with water
A) Water-soluble concentrates (SL)
   10 parts by weight of a compound according to the invention are dissolved in water or in a water-soluble solvent. As an alternative, wetters or other auxiliaries are added. The active compound dissolves upon dilution with water.
B) Dispersible concentrates (DC)
   20 parts by weight of a compound according to the invention are dissolved in cyclohexanone with addition of a dispersant, for example polyvinylpyrrolidone. Dilution with water gives a dispersion.
C) Emulsifiable concentrates (EC)
   15 parts by weight of a compound according to the invention are dissolved in xylene with addition of calcium dodecylbenzenesulfonate and castor oil ethoxylate (in each case 5%). Dilution with water gives an emulsion.
D) Emulsions (EW, EO)
   40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.
E) Suspensions (SC, OD)
   In an agitated ball mill, 20 parts by weight of a compound according to the invention are comminuted with addition of dispersants, wetters and water or an organic solvent to give a fine active compound suspension. Dilution with water gives a stable suspension of the active compound.
F) Water-dispersible granules and water-soluble granules (WG, SG)
   50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.
G) Water-dispersible powders and water-soluble powders (WP, SP)
   75 parts by weight of a compound according to the invention are ground in a rotor-stator mill with addition of dispersants, wetters and silica gel. Dilution with water gives a stable dispersion or solution of the active compound.
      2. Products to be applied undiluted
H) Dustable powders (DP)
   5 parts by weight of a compound according to the invention are ground finely and mixed intimately with 95% of finely divided kaolin. This gives a dustable product.
I) Granules (GR, FG, GG, MG)
   0.5 part by weight of a compound according to the invention is ground finely and associated with 95.5% carriers. Current methods are extrusion, spray-drying or the fluidized bed. This gives granules to be applied undiluted.
J) ULV solutions (UL)
   10 parts by weight of a compound according to the invention are dissolved in an organic solvent, for example xylene. This gives a product to be applied undiluted.

The active compounds can be applied as such, in the form of their formulations or in the application forms prepared therefrom, for example in the form of directly sprayable solutions, powders, suspensions or dispersions, emulsions, oil dispersions, pastes, dusts, compositions for broadcasting, or granules, by spraying, atomizing, dusting, broadcasting or watering. The application forms depend entirely on the intended uses; in any case, they should ensure very fine dispersion of the active compounds according to the invention.

Aqueous use forms can be prepared from emulsion concentrates, pastes or wettable powders (spray powders, oil dispersions) by addition of water. To prepare emulsions, pastes or oil dispersions, the substances can be homogenized in water as such or dissolved in an oil or solvent, by means of wetting agents, tackifiers, dispersants or emulsifiers. However, concentrates comprising active compound, wetting agent, tackifier, dispersant or emulsifier and possibly solvent or oil which are suitable for dilution with water can also be prepared.

The active compound concentrations in the ready-to-use preparations can be varied over a relatively wide range. In general, they are from 0.0001 to 10%, preferably from 0.01 to 1%.

It is also possible to use the active compounds with a high degree of success in the ultra-low-volume method (ULV), it being possible to apply formulations comprising more than 95% by weight of active compound or even the active compound without additives.

Oils of various types, wetting agents, adjuvants, herbicides, fungicides, other pesticides and bactericides can be added to the active compounds, if desired even immediately prior to application (tank mix). These agents can be added to the compositions according to the invention in a weight ratio of 1:10 to 10:1.

The compositions according to the invention in the use form as fungicides may also be present in combination with other active compounds, for example with herbicides, insecticides, growth regulators, fungicides or else with fertilizers. In many cases, mixing of the compounds I, or of the compositions comprising them, in the use form as fungicides with other fungicides results in a broader fungicidal spectrum of activity.

The following list of fungicides, in combination with which the compounds according to the invention can be used, is intended to illustrate the possible combinations, but not to impose any limitation:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl
phenylpyrroles, such as fenpiclonil or fludioxonil,
sulfur
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid
cinnamides and analogous compounds, such as dimethomorph, flumetover or flumorph.

### SYNTHESIS EXAMPLES

The procedures described in the synthesis examples below were used to prepare further compounds I by appropriate modification of the starting compounds. The compounds thus obtained are listed in the tables below, together with physical data.

### Example 1

### Preparation of 5-methyl-6-(2-chloro-4-acetylamino-phenyl)-7-(2-methylbutyl)-1,2,4-triazolo[1,5a]pyrimidine (I-16)

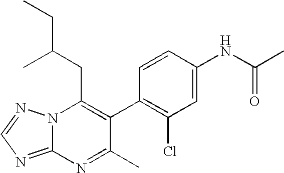

### 1.1. 5-Methyl-6-(2-chloro-4-amino-phenyl)-7-(2-methylbutyl)-1,2,4-triazolo[1,5a]pyrimidine

A mixture of 3 g (8.3 mmol) of 5-methyl-6-(2-chloro-4-nitro-phenyl)-7-(2-methylbutyl)-1,2,4-triazolo[1,5a]pyrimidine (preparation analogously to WO 03/004465), 100 ml of acetic acid, 1 ml of conc. sulfuric acid and 0.5 g of 10% palladium-on-carbon was stirred under an atmosphere of hydrogen overnight.

The reaction mixture was then filtered off with suction through kieselguhr, the ethyl acetate phase was diluted with water and the aqueous phase was extracted three times with methylene chloride. The combined organic phases were washed with NaHCO₃ solution and water until neutral and concentrated. The residue was purified by column chromatography using cyclohexane/ethyl acetate mixture.

This gave 2.1 g (80%) of the title compound 1.1. as a colorless solid.

¹H-NMR (CDCl₃, δ in ppm): 8.45 (s, 1H); 7.0 (d, broad, 1H); 6.9 (s, broad, 1H); 6.7 (d, broad, 1H); 3.6-4.1 (s, very broad, 2H); 3.1 (dd, 0.5H); 2.95 (dd, 0.5H); 2.85 (dd, 0.5H); 2.7 (dd, 0.5H); 2.4 (s, 3H), 2.1 (m, 1H); 1.0-1.4 (m, 2H); 0.7-0.85 (m, 6H)

### 1.2. 5-Methyl-6-(2-chloro-4-acetylamino-phenyl)-7-(2-methyl-butyl)-1,2,4-triazolo[1,5a]pyrimidine

0.25 g (3 mmol) of pyridine and 0.2 g (2.5 mmol) of acetyl chloride were added to a mixture of 0.5 g (1.5 mmol) of 5-methyl-6-(2-chloro-4-amino-phenyl)-7-(2-methylbutyl)-1,2,4-triazolo[1,5a]pyrimidine (example 1.1.) and 10 ml of methylene chloride, and the mixture was stirred at room temperature for about 1 hour.

The reaction mixture was then washed with dilute hydrochloric acid and water and concentrated. The residue obtained was 0.5 g (88%) of the title compound 1.2.

¹H-NMR (CDCl₃, δ in ppm): 8.45 (s, 1H); 8.15 (s, broad, 1H); 7.95 (s, broad, 1H); 7.65 (m, 1H); 7.2 (m, 1H); 3.1 (dd, 0.5H); 2.95 (dd, 0.5H); 2.85 (dd, 0.5H); 2.65 (dd, 0.5H); 2.4 (s, 3H); 2.3 (s, 3H); 2.1 (m, 1H); 1.0-1.35 (m, 1H); 0.7-0.85 (m, 6H)

### Example 2

### 5-Methyl-6-(2,6-difluoro-4-benzylthiophenyl)-7-(2-methylbutyl)-1,2,4-triazolo[1,5a]pyrimidine (I-3)

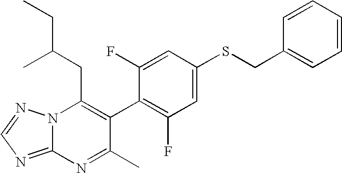

Under an atmosphere of nitrogen, 0.3 g (12.5 mmol) of sodium hydride were added to 1.3 g (10 mmol) of benzyl mercaptan in 50 ml of N-methylpyrrolidone, and the mixture was stirred at room temperature until the evolution of hydrogen had ceased (about 15 min).

3.3 g (10 mmol) of 5-methyl-6-(2,4,6-trifluorophenyl)-7-(2-methylbutyl)-1,2,4-triazolo[1,5a]pyrimidine (preparation analogously to WO 03/004465) were then added, and the mixture was stirred at room temperature for about 2 hours. The reaction mixture was then diluted with water, and the aqueous phase was extracted three times with methyl t-butyl ether. The combined organic phases were washed twice with water and concentrated. The residue obtained was purified by MPLC on silica gel RP-18 using acetonitrile/water mixtures. This gave 2.1 g (50%) of the title compound 2 as a light-yellow, viscous material.

¹H-NMR (CDCl₃, δ in ppm): 8.45 (s, 1H); 7.4 (m, 5H); 6.95 (d, 2H); 4.25 (s, 2H); 3.0 (dd, 1H); 2.75 (dd, 1H); 2.45 (s, 3H); 2.05 (m, 1H); 1.25 (m, 1H); 1.1 (m, 1H); 0.8 (t, 3H); 0.7 (d, 3H)

### Example 3

### 5-Methyl-6-(2,6-difluoro-4-benzylsulfoxyl-phenyl)-7-(2-methylbutyl)-1,2,4-triazolo[1,5a]pyrimidine (I-5)

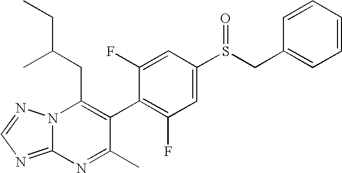

0.5 g (2.2 mmol) of 77% strength m-chloroperbenzoic acid was added to 0.9 g (2 mmol) of 5-methyl-6-(2,6-difluoro-4-benzyl-thiophenyl)-7-(2-methylbutyl)-1,2,4-triazolo[1,5a]pyrimidine (Example 2) in 30 ml of methylene chloride, and the mixture was stirred at room temperature for about 2 hours.

Dilute aqueous sodium hydroxide solution was then added to the reaction mixture, the phases were separated and the organic phase was extracted twice with water. The organic phase was concentrated and the residue was purified by MPLC on silica gel RP-18 using acetonitrile/water mixtures. This gave 0.7 g (77%) of the title compound 3 as a light-yellow oil.

¹H-NMR (CDCl₃, δ in ppm): 8.5 (s, 1H); 7.35 (m, 3H); 7.1 (m, 4H); 4.2 (dd, 2H); 3.05 (dd, 1H); 2.7 (dd, 1H); 2.45 (s, 3H); 2.1 (m, 1H); 1.3 (m, 1H); 1.1 (m, 1H); 0.8 (t, 3H); 0.75 (d, broad, 3H)

### Example 4

### 5-Methyl-6-(2,6-difluoro-4-benzylsulfonyl-phenyl)-7-(2-methylbutyl)-1,2,4-triazolo[1,5a]pyrimidine (I-6)

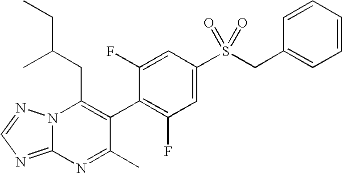

0.3 g (1.3 mmol) of 77% strength m-chloroperbenzoic acid was added to 0.4 g (1 mmol) of 5-methyl-6-(2,6-difluoro-4-benzyl-sulfoxylphenyl)-7-(2-methylbutyl)-1,2,4-triazolo[1,5a]pyrimidine (example 3) in 10 ml of methylene chloride, and the mixture was stirred at room temperature for about 1 hour.

The reaction mixture was then extracted with dilute aqueous sodium hydroxide solution and concentrated, and the residue was purified by column chromatography using cyclohexane/ethyl acetate mixtures. This gave 0.25 g (53%) of the title compound 4 as a light-yellow oil.

¹H-NMR (CDCl₃, δ in ppm): 8.5 (s, 1H); 7.35 (m, 5H); 7.2 (d, 2H); 4.45 (s, 2H); 3.0 (dd, 1H); 2.7 (dd, 1H); 2.4 (s, 3H); 2.1 (m, 1H); 1.3 (m, 1H); 1.1 (m, 1H); 0.8 (t, 3H); 0.7 (d, 3H)

| | |
|---|---|
| | I |
| 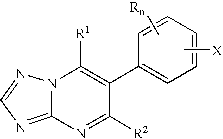 | |
| Table of active compounds | |

| | | | | | Physical data |
|---|---|---|---|---|---|
| No. | R¹ | R² | Rₙ | X | (m.p. [° C.], IR [cm⁻¹], ¹H-NMR [ppm]) |
| I-1 | but-3-enyl | methyl | 2,6-F₂ | 4-S-t-C₄H₉ | 100-102 |
| I-2 | but-3-enyl | methyl | 2,6-F₂ | 4-SO₂-t-C₄H₉ | 17614 178 |
| I-3 | 2-methylbutyl | methyl | 2,6-F₂ | 4-S-benzyl | 8.45 (s, 1H); 4.25 (s, 2H); 2.45 (s, 3H) |
| I-4 | 2-methylbutyl | methyl | 2,6-F₂ | 4-S-t-C₄H₉ | 95-97 |
| I-5 | 2-methylbutyl | methyl | 2,6-F₂ | 4-SO-benzyl | 8.5 (s, 1H); 4.2 (dd, 2H); 2.45 (s, 3H) |
| I-6 | 2-methylbutyl | methyl | 2,6-F₂ | 4-SO₂-benzyl | 8.5 (s, 1H); 4.45 (s, 2H); 2.4 (s, 3H) |
| I-7 | 2-methylbutyl | methyl | 2,6-F₂ | 4-S—CH₃ | 8.45 (s, 1H); 2.6 (s, 3H); 2.45 (s, 3H) |
| I-8 | 2-methylbutyl | methyl | 2,6-F₂ | 4-SO—CH₃ | 8.5 (s, 1H); 2.85 (s, 3H); 2.45 (s, 3H) |
| I-9 | 2-methylbutyl | methyl | 2,6-F₂ | 4-SO₂—CH₃ | 8.5 (s, 1H); 3.25 (s, 3H); 2.5 (s, 3H) |
| I-10 | 2-methylbutyl | methyl | 2,6-F₂ | 4-SO₂-n-C₃H₇ | 8.45 (s, 1H); 3.2 (t, 2H); 2.45 (s, 3H) |
| I-11 | 2-methylbutyl | methyl | 2,6-F₂ | 4-S—C₂H₅ | 8.45 (s, 1H); 3.1 (q, 2H); 2.45 (s, 3H) |
| I-12 | 2-methylbutyl | methyl | 2,6-F₂ | 4-S-n-C₃H₇ | 8.45 (s, 1H); 3.0 (t, 2H); 2.45 (s, 3H) |
| I-13 | 2-methylbutyl | methyl | 2,6-F₂ | 4-SO—C₂H₅ | 8.45 (s, 1H); 7.4 (m, 2H); 2.45 (s, 3H) |
| I-14 | 2-methylbutyl | methyl | 2,6-F₂ | 4-SO₂—C₂H₅ | 122-124 |
| I-15 | 2-methylbutyl | methyl | 2,6-F₂ | 4-SO-n-C₃H₇ | 8.5 (s, 1H); 2.9 (t, 2H); 2.45 (s, 3H) |
| I-16 | 2-methylbutyl | methyl | 2-Cl | 4-NH—CO—CH₃ | 8.45 (s, 1H); 2.4 (s, 3H); 2.3 (s, 3H) |

Examples of the activity against harmful fungi

The fungicidal activity of the compounds of the formula I was demonstrated by the following experiments:

The active compounds were formulated separately as a stock solution comprising 0.25% by weight of active compound in acetone or DMSO. 1% by weight of the emulsifier Uniperol® EL (wetting agent having emulsifying and dispersing action based on ethoxylated alkylphenols) was added to this solution. The stock solutions of the active compounds were diluted with water to the stated concentration.

### Use Examples

### Example 1

### Activity Against Mildew on Cucumber Leaves Caused by Sphaerotheca fuliginea, Protective Application

Leaves of cucumber seedlings of the cultivar “Chinese Snake” which had been grown in pots were, at the cotyledon stage, sprayed to runoff point with an aqueous suspension in the active compound concentration indicated below. 20 hours after the spray coating had dried on, the plants were inoculated with an aqueous spore suspension of mildew of cucumbers (Sphaerotheca fuliginea). The plants were then cultivated in a greenhouse at 20-24° C. and 60-80% relative atmospheric humidity for 7 days. The extent of the mildew development was then determined visually in % infection of the cotyledon area.

In this test, the plants which had been treated with 250 ppm of the compounds I-7 and I-8 showed an infection of ≦20%, whereas the untreated control plants were 90% mildew infected.

### Example 2

### Activity Against Early Blight of Tomato Caused by Alternaria solani

Leaves of potted plants of the cultivar “Golden Princess” were sprayed to runoff point with an aqueous suspension having the concentration of active compound stated below. The next day, the leaves were infected with an aqueous spore suspension of Alternaria solani in a 2% biomalt solution having a density of 0.17×10⁶ spores/ml. The plants were then placed in a water-vapor-saturated chamber at temperatures between 20 and 22° C. After 5 days, the blight on the untreated, but infected control plants had developed to such an extent that the infection could be determined visually in %.

In this test, the plants which had been treated with 250 ppm of the compounds I-4, I-7, I-12 and I-13 showed an infection of ≦30%, whereas the untreated (control) plants were 90% damaged by the fungal infection.

### Example 3

### Activity Against Peronospora of Grape Vines Caused by Plasmopara viticola

Leaves of potted vines were sprayed to runoff point with an aqueous suspension having the concentration of active compound stated below. The next day, the undersides of the leaves were inoculated with an aqueous sporangial suspension of Plasmopara viticola. The vines were initially placed in a water-vapor-saturated chamber at 24° C. for 48 hours and then in a greenhouse at temperatures between 20 and 30° C. for 5 days. After this period of time, the plants were again placed in a humid chamber for 16 hours to promote sporangiophore eruption. The extent of the development of the infection on the undersides of the leaves was then determined visually.

In this test, the plants which had been treated with 250 ppm of the compounds I-4 to I-7 showed an infection of ˜30%, whereas the untreated (control) plants were 80% infected by harmful fungi.

We claim:

1. A triazolopyrimidine of the formula I 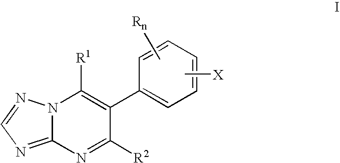
where the index and the substituents are as defined below:
R¹ is C₁-C₁₀-alkyl, C₂-C₁₀-alkenyl, C₂-C₁₀-alkynyl, C₃-C₁₀-cycloalkyl, C₃-C₁₀-cycloalkenyl, phenyl, naphthyl or a five- to ten-membered saturated, partially unsaturated or aromatic heterocycle which is attached via carbon to the triazolopyrimidine and contains one to four heteroatoms from the group consisting of O, N and S,
where R¹ may be partially or fully halogenated or substituted by one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₃-C₆-cycloalkyl, phenyl, naphthyl, a five- to ten-membered saturated, partially unsaturated or aromatic heterocycle which contains one to four heteroatoms from the group consisting of O, N and S,
where these aliphatic, alicyclic or aromatic groups for their part may be partially or fully halogenated or carry one to three groups R^{b}:
R^{b} is halogen, cyano, nitro, hydroxyl, mercapto, amino, carboxyl, aminocarbonyl, aminothiocarbonyl, alkyl, alkenyl, alkynyl, alkenyloxy, alkynyloxy, alkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the abovementioned alkenyl or alkynyl groups in these radicals contain 2 to 8 carbon atoms and the abovementioned groups may be partially or fully halogenated;
and/or one to three of the following radicals:
cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups;
R² is C₁-C₄-alkyl which may be substituted by halogen, cyano, nitro or C₁-C₂-alkoxy;
n is 0 or an integer from 1 to 4;
R is halogen, cyano, C₁-C₆-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₁-C₈-alkoxycarbonyl, C₂-C₁₀-alkenyloxycarbonyl, C₂-C₁₀-alkynyloxycarbonyl, aminocarbonyl, C₁-C₈-alkylaminocarbonyl, di-(C₁-C₈-)alkylaminocarbonyl, C₁-C₈-alkoximinoalkyl, C₂-C₁₀-alkenyloximinoalkyl, C₂-C₁₀-alkynyloximinoalkyl, C₁-C₈-alkylcarbonyl, C₂-C₁₀-alkenylcarbonyl, C₂-C₁₀-alkynylcarbonyl, C₃-C₆-cycloalkylcarbonyl, or a five- to ten-membered saturated, partially unsaturated or aromatic heterocycle which contains one to four heteroatoms from the group consisting of O, N and S;
X is SOₘ—R^{x}, NR^{x}R^{y} or NR^{x}—(C═O)—R^{y};
R^{x}, R^{y}: are: hydrogen, C₁-C₆-alkyl, C₂-C₁₀-alkenyl, C₂-C₁₀-alkynyl, C₃-C₆-cycloalkyl, C₃-C₆-cycloalkenyl, where the above radicals may be partially or fully halogenated or substituted by cyano, C₁-C₄-alkoximino, C₂-C₄-alkenyloximino, C₂-C₄-alkynyloximino or C₁-C₄-alkoxy;
m is 0 or an integer 1 to 3.

2. A triazolopyrimidine of the formula I′ 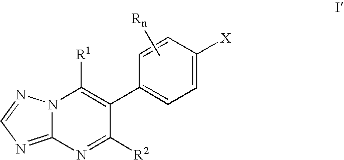
where the index and the substituents are as defined below:
R¹ is C₃-C₈-alkyl, C₃-C₈-alkenyl, C₃-C₈-alkynyl, C₃-C₆-cycloalkyl, C₅-C₆-cycloalkenyl; where R¹ may be partially or fully halogenated or substituted by one to four identical or different groups R^{a}:
R^{a} is halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino, C₂-C₆-alkynyloximino, C₃-C₆-cycloalkyl, C₅-C₆-cycloalkenyl, where the aliphatic or alicyclic groups for their part may be partially or fully halogenated or carry one to three groups R^{b}:
R^{b} is halogen, cyano, C₁-C₆-alkyl, C₁-C₆-haloalkyl, C₂-C₆-alkenyl, C₂-C₆-alkynyl, C₁-C₆-alkylcarbonyl, C₁-C₆-haloalkylcarbonyl or C₁-C₆-alkoxy;
R² is C₁-C₄-alkyl which may be substituted by halogen;
n is an integer from 0 to 2;
R is fluorine, chlorine, bromine, cyano, C₁-C₆-alkyl, C₁-C₆-alkoxy;
X is SO—R^{x}, SO₂—R^{x} or NR^{x}—(C═O)—R^{y};
R^{x}, R^{Y} are: hydrogen, C₁-C₆-alkyl, C₂-C₆-alkenyl or C₃-C₆-cycloalkyl, where the above radicals may be partially or fully halogenated.

3. A process for preparing compounds of the formula I as claimed in claim 1 or 2 which comprises reacting 5-aminotriazole of the formula II 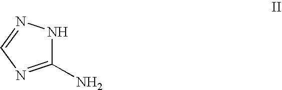
with dicarbonyl compounds of the formula III 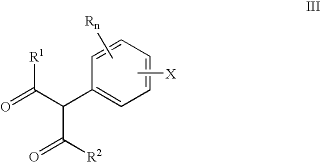
where the substituents R, X, R¹ and R² and the index n are as defined in claim 1.

4. A dicarbonyl compound of the formula III, which is defined in claim 3.

5. A composition suitable for controlling harmful fungi, comprising a solid or liquid carrier and a compound of the formula I as claimed in claim 1.

6. The use of the compounds I as claimed in claim 1 for preparing a composition suitable for controlling harmful fungi.

7. A method for controlling harmful fungi, which comprises treating the fungi or the materials, plants, the soil or seeds to be protected against fungal attack with an effective amount of a compound of the formula I as claimed in claim 1.
